(19) United States
(12) Patent Application Publication
(10) Pub. No.: US 2015/0175747 A1
(43) Pub. Date: Jun. 25, 2015

(54) Highly Emissive Far-Red/Near-Infrared Fluorescent Conjugated Polymer-Based Nanoparticles

(71) Applicant: National University of Singapore, Singapore (SG)

(72) Inventors: Bin Liu, Singapore (SG); Jie Liu, Singapore (SG); Dan Ding, Singapore (SG); Junlong Geng, Singapore (SG); Lun-de Liao, Singapore (SG)

(21) Appl. No.: 14/415,315

(22) PCT Filed: Jul. 25, 2013
(86) PCT No.: PCT/SG2013/000308

§ 371 Date: Jan. 16, 2015

Related U.S. Application Data
(60) Provisional application No. 61/675,570, filed on Jul. 25, 2012.
(60) Provisional application No. 61/845,672, filed on Jul. 12, 2013.

(51) Int. Cl. C08G 75/06; A61B 5/00; C08K 5/521; C08L 67/04; C08L 81/02; C08L 89/00

(52) U.S. Cl. CPC C08G 75/06; C08L 81/02; C08L 89/00; C08K 5/521; C08L 67/04; A61B 5/0095

(57) ABSTRACT

A series of conjugated polymer-based nanoparticles having far-red/near infrared emission ranges are disclosed. Cross coupling methods to prepare the conjugated polymers and methods of nanoparticle preparation are also discussed. The conjugated polymer nanoparticles are used as FR/NIR fluorescent probes in in vitro and in vivo biosensing and bioimaging applications, and are also used in photoacoustic imaging as contrast agents. Finally, use of the conjugated polymer nanoparticles in photothermal therapy is described.

### RELATED APPLICATIONS

This application claims the benefit of U.S. Provisional Application No. 61/675,570, filed on Jul. 25, 2012, and U.S. Provisional Application No. 61/845,672, filed on Jul. 12, 2013. The entire teachings of the above applications are incorporated herein by reference.

### BACKGROUND OF THE INVENTION

Development of reliable fluorescent probes with high sensitivity and selectivity for biosensing and bioimaging applications is of central importance. Since the far-red/near-infrared (FR/NIR) region (650-1000 nm) offers a unique interrogation window for biological applications with minimal interferential absorption, low biological autofluorescence, and high tissue penetration, FR/NIR fluorescent probes have attracted great interest in the multidisciplinary field of biology, chemistry and materials science.[1] So far, various materials including organic fluorophores, fluorescent proteins and inorganic semiconductor quantum dots (QDs) have been widely exploited for FR/NIR fluorescence biosensing and bioimaging. Organic fluorophores and fluorescent proteins, however, suffer from limited molar absorptivity and poor photostability, while inorganic QDs are highly cytotoxic in an oxidative environment, which have greatly limited the scope of their biological applications.[2] Exploration of a new generation of FR/NIR fluorescent probes with high fluorescence, strong photobleaching resistance, as well as low cyto- and systemic toxicity is highly desirable for in vitro and in vivo sensing and imaging applications.

In addition to fluorescence-based bioimaging, photoacoustic microscopy (PAM) has emerged as a promising technique in biological imaging due to its noninvasive, high resolution and deep penetration characteristics. [3-6] Photoacoustic (PA) imaging is built on the PA effect, which involves light absorption by target objects, transient thermoelastic expansion and subsequent generation of ultrasonic waves.[7,8] PA imaging contrast generally relies on the optical absorption of the target substances in the excitation wavelength. Based on the intrinsic optical contrasts in biological systems, numerous biological applications including have been achieved, including visualizing blood vessel structures,[9] studying brain hemodynamic changes,[10-13] and imaging tumor angiogenesis[14]. Unfortunately, most intrinsic optical contrasts, such as hemoglobin and deoxy-hemoglobin, absorb light in the visible spectral region, a region having overwhelming light scattering in biological tissues, which results in limited sensitivity and resolution.[3] In addition, many biological objects or disease signal molecules do not show PA contrast due to their low extinction coefficient, which hampers specific detections with their inherent PA signals. However, implementation of an exogenous contrast agents serves to enhance the sensitivity of PA imaging by adjusting the absorption region in near infrared (NIR) spectral region to reduce the scattering interference, and also to target the specific biological objects to generate sufficient PA signals for accurate analysis.[9,15-18]

Exogenous contrast agents, such as optically absorptive organic dyes, metal and carbonous nanomaterials and fluorescent proteins, have been applied in biological systems and shown improved PA contrasts in in vivo studies.[18-21] In particular, organic dye based photoacoustic contrast reagents, such as indocyanine green (ICG) or IRDye 800 conjugated to cyclo(Lys-Arg-Gly-Asp-Phe), are biocompatible and biodegradable in biological environment.[9,14,16,22,23] Both ICG and IRDye 820 are also fluorescent dyes. Typically in the art, it is believed that two factors determining the strength of PA signals of fluorescent material-based exogenous contrast reagents are (1) a large intrinsic extinction coefficient and (2) high nonradiative quantum yield, defined as (1−quantum yield).[3,21] Both the extinction coefficient and nonradiative quantum yield of a fluorophore can be adjusted by synthetic design, permitting the experimentalist to fine tune a fluorophore for performance in PA imaging. Due to light scattering in the visible spectral region, there is a need to develop photoacoustic contrast reagents having absorption in the FR/NIR region.

Conjugated polymers (CPs) are macromolecules with π-conjugated backbones, which allow the formation of excitons to facilitate photo- and electroluminescence.[24-27] CPs combine semiconducting properties and light harvesting abilities, which have made them an important class of optoelectronic materials in applications spanning from light-emitting diodes to field-effect transistors and photovoltaic devices. In addition, their large absorption coefficient, light-harvesting properties and signal amplification effects open up opportunities for highly sensitive chemical and biological sensing. Most recently, great research interest has also been focused on the application of CPs for bioimaging due to their good photostability and low cytotoxicity, which makes them very promising materials for next generation fluorescent probes. [28]

In the past years, various CPs with emission spanning from ultraviolet through visible to near-infrared, have been developed, which are readily tuned by employing copolymerization or varying linkage bonds. However, most reported CPs exhibit poor water solubility due to the hydrophobic aromatic backbones. As a result, CPs usually demonstrate low photoluminescence (PL) quantum efficiency, induced by strong tendency to self-assemble into aggregates in aqueous solution. In general, the PL quantum efficiency decreases sharply with changing the emission from blue to red. Quantum yields of the most reported CPs with FR/NIR emission are below 2%.[29] In spite of the unique advantages of CPs with FR/NIR fluorescence in biosensing and bioimaging applications, there is still a challenge to design CP-based probes with high molar extinction coefficient at FR/NIR wavelength and high quantum yield.

### SUMMARY OF THE INVENTION

The present invention relates to the synthesis and application of highly emissive FR/NIR light-emitting CP-based nanoparticles (NPs). Conjugated polymers with FR/NIR fluorescence were prepared by Suzuki or Stille polycondensation, which was followed by click chemistry to incorporate hydrophilic side chains to self-assemble into CP NPs. Within the conjugated backbones, two narrow band gap units were employed as donor and acceptor segments to achieve Förster Resonance Energy Transfer (FRET). In addition, the CPs were also encapsulated into biocompatible matrix to fabricate CP-loaded NPs. The excess functional groups in the matrix allow further surface functionalization of the NPs with targeting ligands.

The invention relates to conjugated polymers of formula (I):

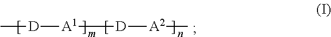

wherein: D is a conjugated system of one or more optionally substituted aromatic or heteroaromatic groups comprising:

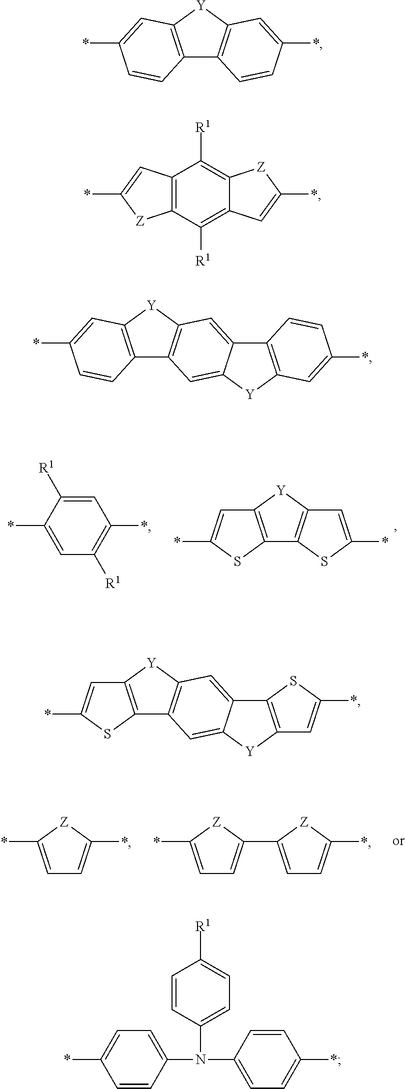

A¹ is a conjugated system of one or more optionally substituted aromatic or heteroaromatic groups;
A² is a conjugated system of one or more optionally substituted aromatic or heteroaromatic groups;
m is an integer from 1 to 100;
n is an integer from 0 to 100;

### Y is C(R¹)₂, Si(R¹)₂, or NR¹;

### Z is O, S or Se;

R¹ is independently H, (CH₂)ₚQ, (OCH₂CH₂)ₚQ,

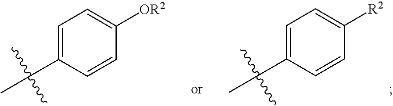

p is an integer ranging from 1 to 24;
R² is (C₁-C₁₂)alkyl, (C₆-C₁₄)aryl, or (C₁-C₁₀)alkoxy(C₁-C₁₂)alkyl;
Q is independently CH₃, H, COOH, NH₂, NH₃⁺, N₃, SH, SO₃Na, PO₃Na, or

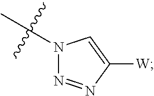

W is independently a fluorophore, a bioconjugate, a (C₁-C₁₀₀)alkyl group bound to a fluorophore or a bioconjugate, or a polyethyleneoxide group bound to a fluorophore or a bioconjugate;
* is the point of attachment to A¹, A², or a polymeric unit;
for n greater than or equal to 1, the energy band gap of A¹ is larger than the energy band gap of A²; and
further wherein the energy band gap of D is larger than the energy band gap of A¹ and the energy band gap of A².

The conjugated polymer of the present invention self-assemble into nanoparticles, or alternately are encapsulated into a biocompatible matrix comprising polyethylene glycol, polyethylene glycol conjugated to 1,2-distearoyl-sn-glycero-3-phosphoethanolamine, bovine serum albumin (BSA) protein, poly(lactic-co-glycolic acid) (PLGA) block copolymers, collagens or lipids.

In aspects of the invention, substituents A¹ and A² in Formula (I) comprise

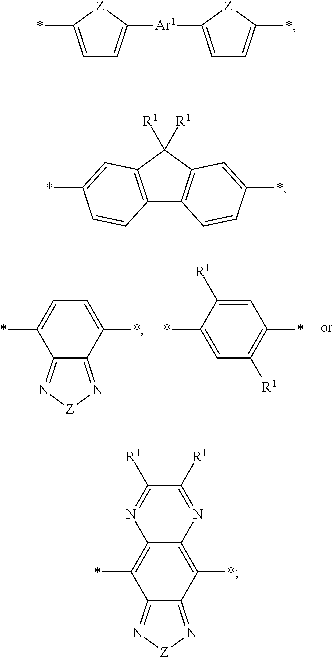

wherein:

### Ar¹ is

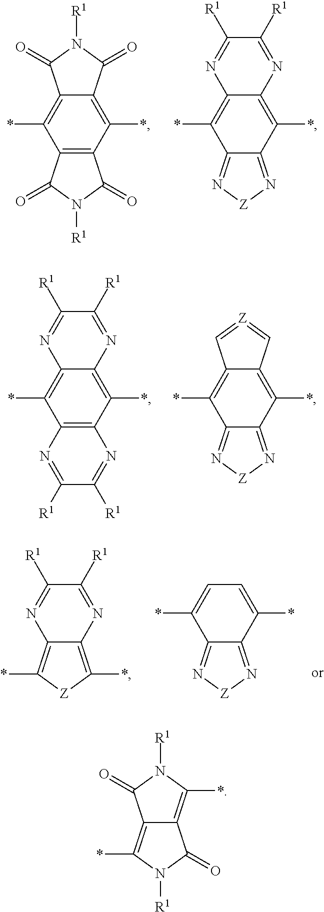

Substituent W on the optional triazole of R¹ comprises

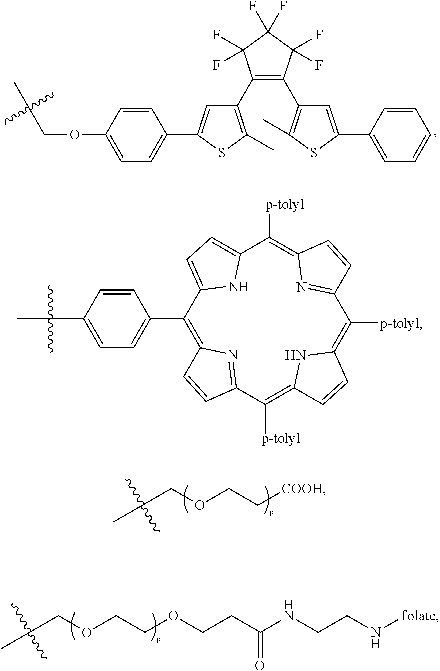

an amine-labeled cyclic peptide, an oligonucleotide, an acyclic peptide, or a protein, wherein v is an integer ranging from 0 to 45.

Preferred embodiments of the invention include the conjugated polymers CP1, PFTTQ, and PIDT-TTQ.

The present invention also relates to methods for making conjugated polymer based nanoparticles comprising a Suzuki or Stille-type cross coupling reaction, comprising reacting a cross coupling partner such as an organoborate or an organostannane containing donor group D with a dihalide containing A¹, and for compounds for which n is not equal to zero, a second dihalide containing A² in the presence of a transition metal catalyst to produce the conjugated polymer. The conjugated polymer is then solubilized or suspended in aqueous solution to self-assemble into nanoparticles.

The CP-based NPs of the invention show high quantum yields in water (21-32%) and high thermal and photostability. Cellular and in vivo imaging studies reveal that these CP-based NPs can be used as fluorescent probes in bioimaging applications, and particularly have shown the utility of compounds of Formula (I) in cancer cell staining. The present invention further relates to the use of the CP NPs described herein as photosensitizers in photodynamic therapy.

The CPs described herein also demonstrate high PA contrast and good photothermal therapy performance. The synthesized CP NPs show higher photoacoustic signal than do conventional agents of indocyanine green (ICG) and polyhydroxyfullerene (PHF). The larger absorption coefficient and low fluorescence quantum yield of CPs contribute to their high PA intensities. Additionally, the obtained CP NPs show high heat generation capacity under NIR light irradiation, which is effective for cancer cell treatment in mere minutes. Surface functionalization of CP NPs enables their ability to target biological species, which enhances the application of CP NPs in tumor treatment. Such photoacoustic probes have the potential for application in high resolution imaging in tissues with a penetration depth in mm to cm.

The present invention also describes methods for photoacoustic imaging of a target utilizing a compound of Formula (I), comprising incubating a target with a polymer of Formula (I) to form an incubated mixture; irradiating the mixture with a pulsed laser, wherein the pulsed laser optically excites the polymer, to generate thermally-induced acoustic waves, wherein the acoustic waves result from energy emission from the excited polymer; detecting the thermally-induced acoustic waves with ultrasound; and translating the acoustic waves detected by ultrasound into an image of the target. In addition, the synthesized CP NPs generate heat upon NIR light absorption.

Therefore, another aspect of the invention relates to the use of the CP NPs described herein as a therapeutic agent for use in photothermal therapy. The present invention further provides a method for photothermal ablation of a cancer cell, comprising: incubating a cancer cell with a conjugated polymer nanoparticle of formula (I) to form an incubated mixture; and irradiating the mixture with a laser, wherein the polymer absorbs energy from laser irradiation and converts the energy to heat, thereby causing ablation of a cancer cell.

The present invention also relates to low molecular weight conjugated molecules of the formula (II):

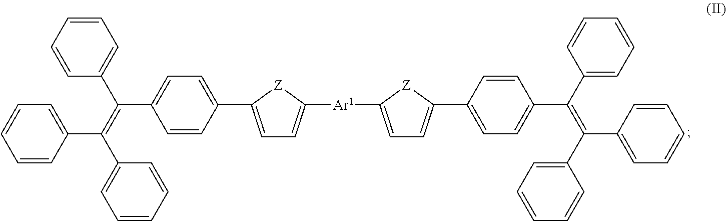

wherein:

### Ar¹ is

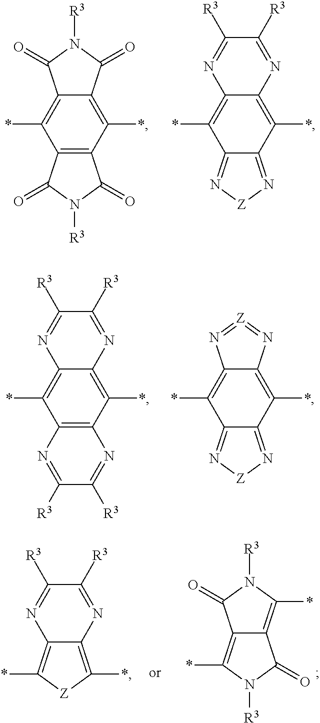

R³ is independently H, (CH₂)ₚCH₃, (OCH₂CH₂)ₚCH₃,

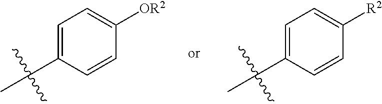

p is an integer ranging from 1 to 24;
R² is (C₁-C₁₂)alkyl, (C₆-C₁₄)aryl, or (C₁-C₁₀)alkoxy(C₁-C₁₂)alkyl; and
* is the point of attachment to A¹, A², or a polymeric unit.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 shows hydrodynamic diameter distribution of PFBDDBT10-PEG₁₀₀₀-COOH in water. The particles were prepared by adding 2 mL of PFBDDBT10-PEG₁₀₀₀-COOH DMSO solution with concentration of 0.5 mg/mL (a), 0.25 mg/mL (b) and 0.17 mg/mL (c) into 10 mL Milli-Q water under sonication, respectively.

FIGS. 2a and 2b show the (a) UV-vis and (b) PL spectra of PFDBT10-PEG₁₀₀₀-COOH (black), PFBTDBT10-PEG₁₀₀₀-COOH (red) and PFBDDBT10-PEG₁₀₀₀-COOH (blue) NPs in water, respectively. The excitation occurred at maximum absorption wavelength. The insert of FIG. 2b shows photography pictures of PFDBT10-PEG₁₀₀₀-COOH, PFBTDBT10-PEG₁₀₀₀-COOH and PFBDDBT10-PEG₁₀₀₀-COOH solutions (from left to right) under illumination at 365 nm.

FIG. 3a shows PL spectra of PFBDDBT10-PEG₁₀₀₀-COOH in the presence of BSA with the concentration of BSA ranging from 0 to 0.25 μM at intervals of 0.05 μM in 150 mM PBS, with excitation at 488 nm. The arrow indicates the increase of the concentration of BSA. FIG. 3b shows a plot of fluorescence quantum yields for PFDBT10-PEG₁₀₀₀-COOH, PFBTDBT10-PEG₁₀₀₀-COOH and PFBDDBT10-PEG₁₀₀₀-COOH in water, respectively. The CP NPs were incubated in PBS/BSA (150 mM/0.25 μM) mixture at 37° C. for 0, 1, 2 and 3 days.

FIG. 4 shows physical characteristics of formulations of PFBTDBT10 nanoparticles. FIG. 4a shows a size distribution of CP-loaded DSPE-PEG NPs, FIG. 4b shows a TEM image of the nanoparticles, while FIG. 4c shows UV-Vis and PL spectra of CP-loaded DSPE-PEG-FA nanoparticles. The chemical structure of PFBTDBT10 is shown in FIG. 4d.

FIGS. 5a and 5b show (a) PL intensity changes of CP-loaded DSPE-PEG-FA NPs, Alexa Fluor 555 and Rhodamine 6G when incubating in PBS buffer at 37° C. for 7 days, and (b) Hydrodynamic size change of the CP-loaded DSPE-PEG-FA NPs when incubating in PBS buffer at 37° C. for 7 days.

FIG. 6 shows confocal fluorescence images of MCF-7 breast cancer cells after 2 h incubation with (a) CP-loaded DSPE-PEG-FA NPs and (b) NPs without folate, respectively. Confocal fluorescence images of NIH/3T3 fibroblast cells after 2 h incubation with (c) CP-loaded DSPE-PEG-FA NPs and (d) NPs without folate, respectively. The cellular nuclei were stained with 4,6-diamidino-2-phenylindole (DAPI).

FIG. 7 shows photostability comparisons among CP-loaded DSPE-PEG-FA NPs, Alexa Fluor 555 and Rhodamine 6G upon continuous laser excitation at 543 nm for 0-10 min. I₀ is the initial fluorescence intensity; I is the fluorescence intensity of the sample at various time points.

FIG. 8a shows blood circulation curves after intravenous injection of CP-loaded DSPE-PEG NPs with and without folate functionalization, respectively.

FIG. 8a plots percent injection dosing in blood against time. FIGS. 8b and 8c show in vivo non-invasive fluorescence imaging of H22 tumor-bearing mice after intravenous injection of CP-loaded DSPE-PEG NPs with (FIG. 8b) and without folate functionalization (FIG. 8c), respectively.

FIG. 9 shows ex vivo fluorescence images of various organs at 24 h post intravenous injection of CP-loaded DSPE-PEG NPs with (a) and without folate functionalization (b), respectively. (c) Biodistribution analysis of CP-loaded DSPE-PEG NPs with and without folate functionalization in H22 tumor-bearing mice, respectively, at 24 h post administration. FIG. 9c plots average PL intensity in a.u. against tumor, spleen, liver, heart, lung, stomach, kidney, brain and intestine.

FIG. 10a shows body weight changes of the mice with various treatments indicated plotted against time. FIG. 10b shows typical images of H&E-stained liver and spleen slices from H22 tumor-bearing mice treated with different protocols. The scale bar is 100 μm.

FIG. 11 shows the UV-vis and PL spectra of CP1 nanoparticles (NPs) in water. The absorption of the CP1 nanoparticles has two absorption peaks centered at 455 and 563 nm, which correspond to π-π* transition of the conjugated backbone and charge transfer state, respectively. The NPs exhibits a PL spectrum with an emission peak at 685 nm. The quantum yield (QY) of the NPs based on CP1 was measured to be 7±1%, using rhodamine 6G in methanol as reference (QY=95%).

FIG. 12a shows metabolic viability of MCF-7 breast cancer cells after incubation with PFBDDBT10-PEG₁₀₀₀-FA NPs with concentration of 2, 10 and 20 μM for 24 h, 48 h and 48 h, respectively; FIG. 12b depicts the photostability for PFBDDBT10-PEG₁₀₀₀-FA NPs in MCF-7 cancer cells upon continuous laser excitation at 488 nm with laser power of 2.5 mW from 0 to 10 min. I₀ is the initial fluorescence intensity and I is the fluorescence intensity of sample at various time points after continuous scanning.

FIG. 13 shows the UV and PL spectra of PFBDDBT10-PEG1000-FA in water.

FIG. 14 shows the UV-Vis absorption spectra of PFTTQ and PIDT-TTQ NPs in water. PFTTQ has two absorption peaks located at ˜430 and 775 nm, which were attributed to π-π* transition of the conjugated backbone and charge transfer state, respectively. PIDT-TTQ NPs show a broad UV-vis-NIR absorption band from 620 nm to 1100 nm. Both PFTTQ and PIDT-TTQ NPs have strong absorption at 800 nm, where the light has deep penetration in biological tissue.

FIG. 15 shows the experimental setup of the photoacoustic microscopy system.

FIG. 16a shows PA intensity measurements for PFTTQ NPs, PIDT-TTQ NPs, ICG and PHF with the same mass concentrations of 1 mg/mL. FIG. 16b shows the respective PA images of (1) PFTTG NPs, (2) PIDT-TTQ NPs, (3) ICG and (4) PHF. All images share the same scale bar of 100 μm.

FIG. 17 shows the temperature evolution of PFTTQ NPs, PIDT-TTQ NPs and water under 800 nm laser irradiation at a power density of 1.5 W/cm².

FIG. 18 shows relative viabilities of MCF-7 breast cancer cells after PFTTQ NP induced photothermal ablation at different laser power densities.

FIG. 19 shows fluorescence images of PI-stained MCF-7 cancer cells with and without PFTTQ NP incubation after being exposed to 800 nm laser at different power densities. All images share the same scale bar of 200 m.

### DETAILED DESCRIPTION OF THE INVENTION

A description of example embodiments of the invention follows.

CP-based nanoparticles (NPs) with ultrahigh quantum yields in water (from about 21 to about 32%) were developed. The strategy of using a combination of electron-rich and electron-deficient moieties to form alternating donor-acceptor (D-A) backbone structures is employed to develop CPs with FR/NIR emission. One challenge with conjugated polymers with D-A backbones is that, because they possess intramolecular charge transfer, their fluorescence is significantly quenched when in a water medium. To allow for an enhanced quantum yield while increasing the water solubility of CPs, two strategies are employed in this invention. One is to attach hydrophilic side chains to the CP backbone, which is followed by self-assembly into conjugated polymer nanoparticles (CP NPs) and the other is to prepare CP-loaded NPs through nano-precipitation or encapsulation. In addition, the feasibility and advantages of CP-based NPs for biosensing, bioimaging, photoacoustic imaging and photothermal therapy applications are demonstrated.

Scheme 1 shows a general procedure for synthesis of the conjugated polymers of the present invention. Generally, the conjugated polymers contain two components: FR/NIR light-emitting conjugated backbones and optionally functionalized side chains. Within the conjugated backbones, narrow band gap units are employed as donor (D) and acceptor (A¹ and optionally A²) segments, wherein the energy band gap of D is larger than the energy band gap (E_{g}) of the acceptor segments. When the donor segments are excited, fluorescence resonance energy transfer (FRET) occurs from the donor segments to the acceptor segments, thus realizing FR/NIR emission. The absorption wavelength of final CPs is adjusted by changing the donor unit. Similarly, the emission of the final CPs can be tuned by changing the acceptor unit. In some embodiments of the invention, FRET occurs between A¹ and A², wherein the energy band gap of A¹ is larger than that of A². In yet further embodiments of the invention, D and A¹ are identical, and therefore, no FRET occurs.

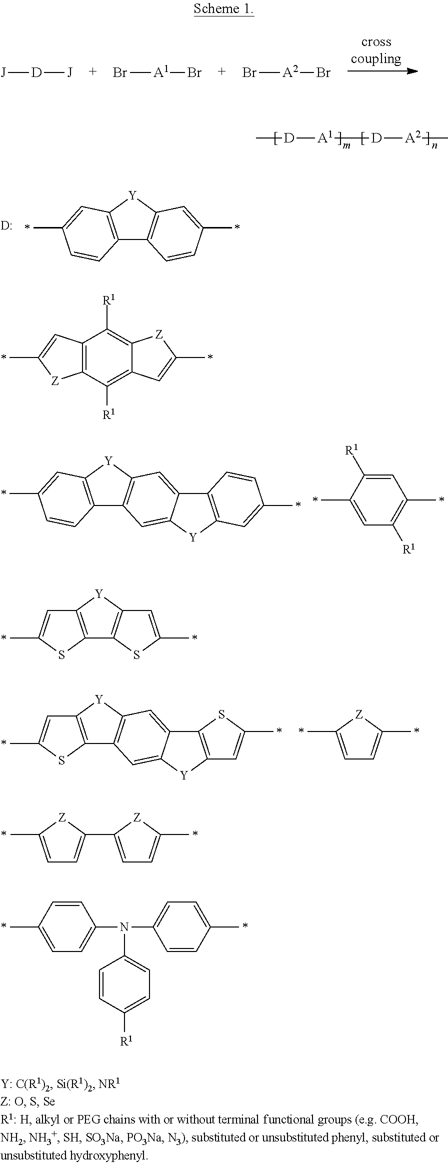

As shown in Scheme 1, the conjugated polymer of Formula (I) is synthesized by a cross coupling polymerization reaction, preferably a Stille or Suzuki cross coupling reaction. The donor segment D is a conjugated system of one or more optionally substituted aromatic or heteroaromatic rings. Possible structures of D are shown in Scheme 1. The cross coupling partner containing D is generally boron- or tin-based. In some aspects of the invention, the cross coupling partner is a stannane (i.e. J is, e.g., SnBu₃), a boronic acid (i.e. J is B(OH)₂), a boronic acid ester (i.e. J is, e.g. B(OCH₃)₂, B(pin):

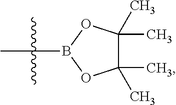 [SMILES: *B1OC(C)(C)C(C)(C)O1]

### B(cat):

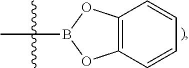 [SMILES: *B1Oc2ccccc2O1]

or a trifluoroborate (i.e. J is, e.g. BF₃K). The cross coupling reaction occurs with one or more dihalide coupling partners, Br-A¹-Br and optionally Br-A²-Br. In some embodiments of the invention, the bromine in the dihalide coupling partner is replaced by a chloride or an iodide. The acceptor segments A¹ and A² are independently conjugated systems of one or more optionally substituted aromatic or heteroaromatic rings.

In Scheme 1, m and n are integers that indicate the number of repeat units in the conjugated polymer, as well as the relative ratio of the constituent functional groups. In certain aspects of the invention, m is an integer ranging from 1 to 100 and is an integer ranging from 0 to 100. In preferred aspects of the invention, m is an integer ranging from 1 to 50 and is an integer ranging from 0 to 50, and in more preferred aspects of the invention, m is an integer ranging from 1 to 20 and is an integer ranging from 0 to 20. Furthermore, the conjugated polymers of the invention are random copolymers.

It is a versatile strategy to synthesize FR/NIR fluorescent CPs with various functional groups at the end of side chains R. In some embodiments of the invention, the side chains of the polymers are alkyl or polyethylene glycol side chains that are optionally modified to have functional groups that include carboxylic acid, amino, protonated amino, phosphate, azide, thiol, maleimide, succinimide or sulfate groups. In further embodiments of the invention, the side chain of the polymer contains an azide that reacts with a substituted alkyne through click chemistry to form a 1,2,3-triazole, which is functionalized by a ligand, such as a peptide, an aptamer or a fluorophore, which will make CPs into probes for specific biological applications. In some embodiments of the invention, the triazole is functionalized by a ligand comprising an acyclic peptide, an amine-labeled cyclic peptide, an oligonucleotide, a protein or a bioconjugate. Specific examples of such conjugated polymers are discussed herein. The length of the alkyl or polyethylene glycol side chains is from 1 to 24 repeat units, or more preferably from 1 to 10 repeat units, wherein a repeat unit for alkyl is —CH₂— and a repeat unit for polyethylene glycol is —CH₂CH₂O—.

In another aspect of the invention, the side chains of the conjugated polymers are substituted or unsubstituted phenyl, or substituted or unsubstituted hydroxyphenyl. In such embodiments, phenyl is optionally substituted at any position by one or more substituents selected from (C₁-C₁₂)alkyl, (C₆-C₁₄)aryl or (C₁-C₁₀)alkoxy(C₁-C₁₂)alkyl, or preferably by C₁-C₆ alkyl. Hydroxyphenyl is optionally substituted on the hydroxy group (i.e. a covalent bond is formed between the oxygen of the hydroxy group and the optional substituent) by one or more substituents selected from (C₁-C₁₂)alkyl, (C₆-C₁₄)aryl or (C₁-C₁₀)alkoxy(C₁-C₂)alkyl, or preferably by C₁-C₆ alkyl.

In some embodiments of the invention, A¹, and A² if A² is present, are conjugated systems of one or more optionally substituted aromatic or heteroaromatic rings, which include the following structures:

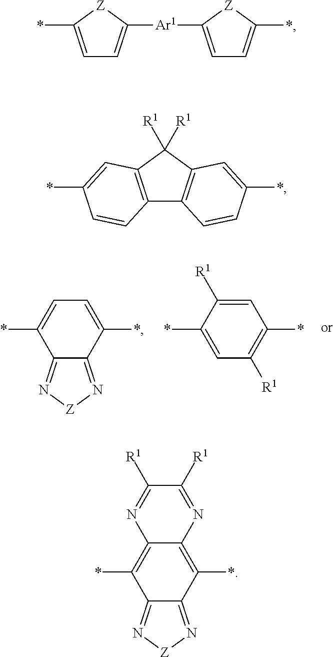

In the preceding structures, Ar¹ represents:

Ar¹ is

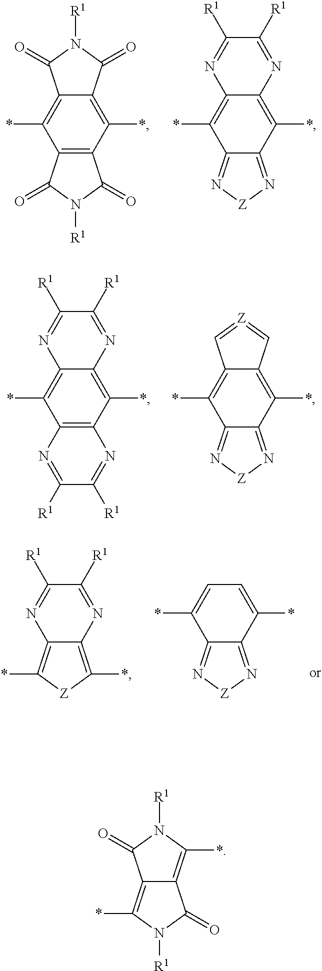

For each of the preceding conjugated ring systems, Z may be O, Se or S, and R¹ is H, alkyl or PEG chains with or without terminal functional groups (e.g. COOH, NH₂, NH₃⁺, SH, SO₃Na, PO₃Na, N₃), substituted or unsubstituted phenyl, substituted or unsubstituted hydroxyphenyl, and additionally can have any of the additional substitution (e.g. functionalized triazole) or conjugation described for R¹ above.

In certain embodiments of the invention, the invention does not include conjugated polymers of the following structure:

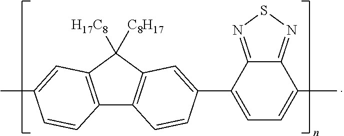

In certain embodiments, the conjugated polymers of the invention emit light in the far red/infrared (FR/IR) range. In other embodiments, the conjugated polymers emit light in the near IR range.

Scheme 2 shows a representative example of molecular design and synthesis. As shown in Scheme 2, P1 was synthesized by a cross coupling polycondensation using a cross-coupling partner. In some embodiments of the invention, the cross coupling partner is a stannane, such as a trimethylstannane or a tributylstannane. In preferred embodiments of the invention, the cross coupling partner is a boron-based coupling partner, such as a boronic acid, a boronic acid pinacol ester, a boronic acid catechol ester, or a trifluoroborate.

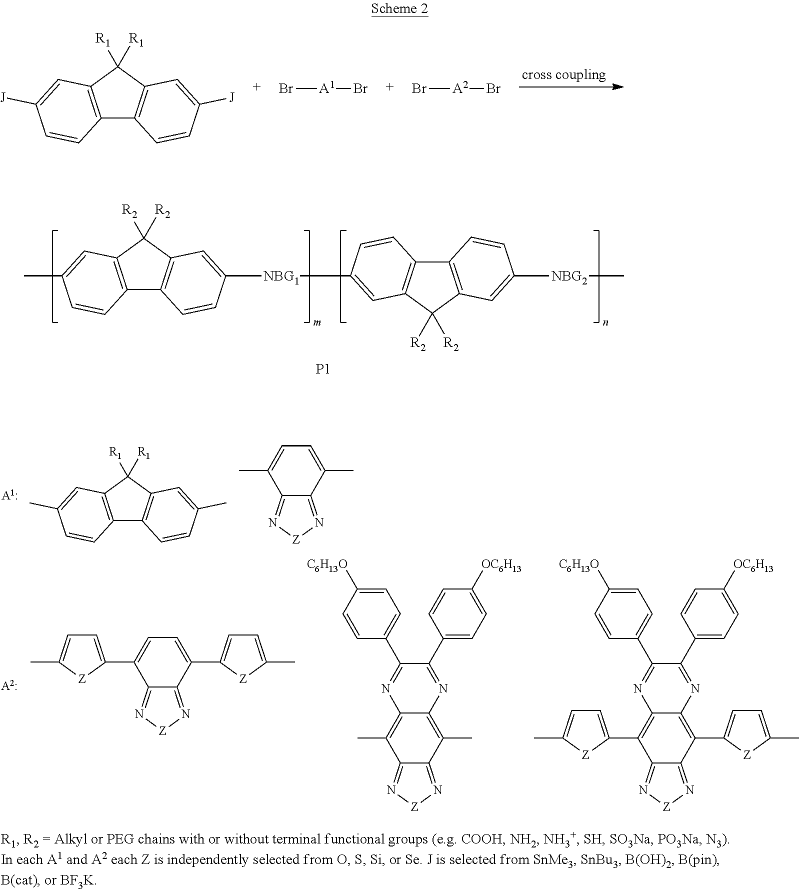

In Scheme 2, m and n are integers that indicate the number of repeat units in the conjugated polymer, as well as the relative ratio of the constituent functional groups. In certain aspects of the invention, m and n are each integers independently ranging from 0 to 100. In preferred aspects of the invention, m and n are each integers independently ranging from 0 to 50, and in more preferred aspects of the invention, m and n are each integers independently ranging from 0 to 20. Furthermore, the conjugated polymers of the invention are random copolymers.

In another embodiment of the invention, the conjugated polymer is synthesized via Suzuki or Stille polycondensation to yield compounds CP1, Poly[9,9-bis(4-(2-ethylhexyl)phenyl)fluorene-alt-co-6,7-bis(4-(hexyloxy)phenyl)-4,9-di(thiophen-2-yl)thiadiazoloquinoxaline](PFTTQ) and Poly[(4,4,9,9-tetrakis(4-(octyloxy)phenyl)-4,9-dihydro-s-indacenol[1,2-b:5,6-b]dithiophene-2,7-diyl)-alt-co-4,9-bis(thiophen-2-yl)-6,7-bis(4-(hexyloxy)phenyl)-[1,2,5]thiadiazolo[3,4-g]quinoxaline](PIDT-TTQ), as shown in Scheme 3.

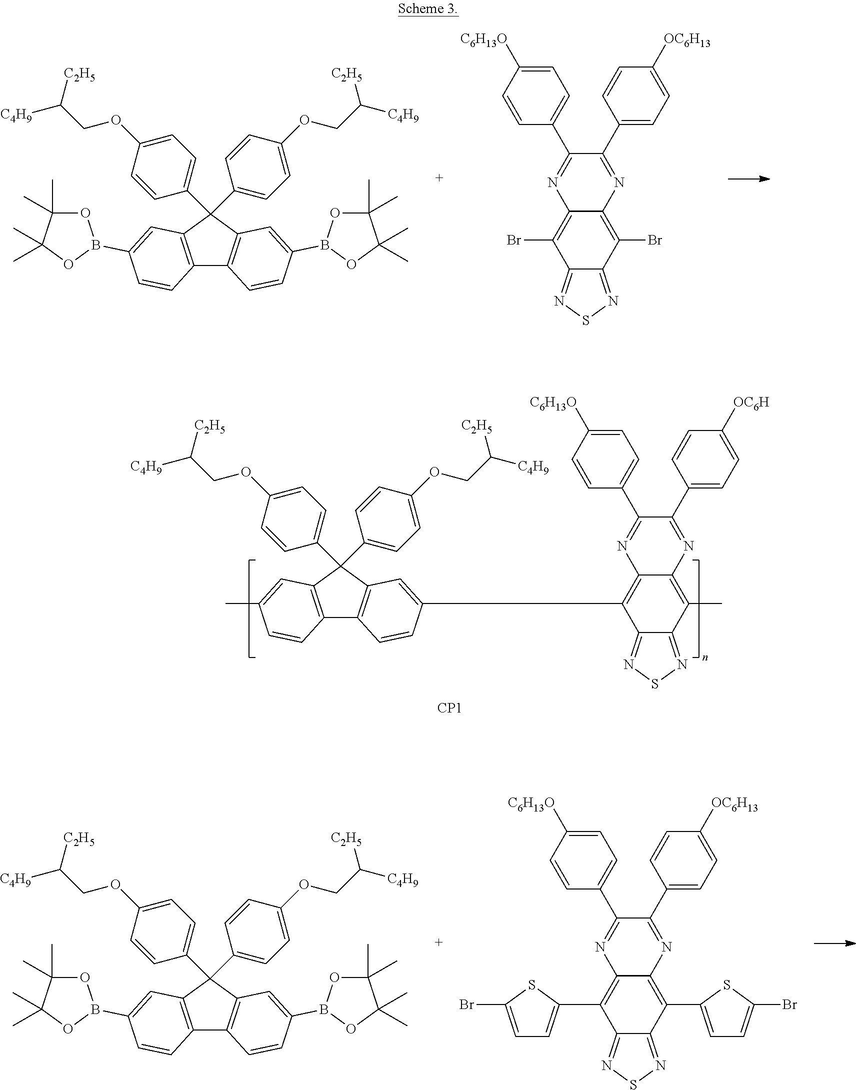

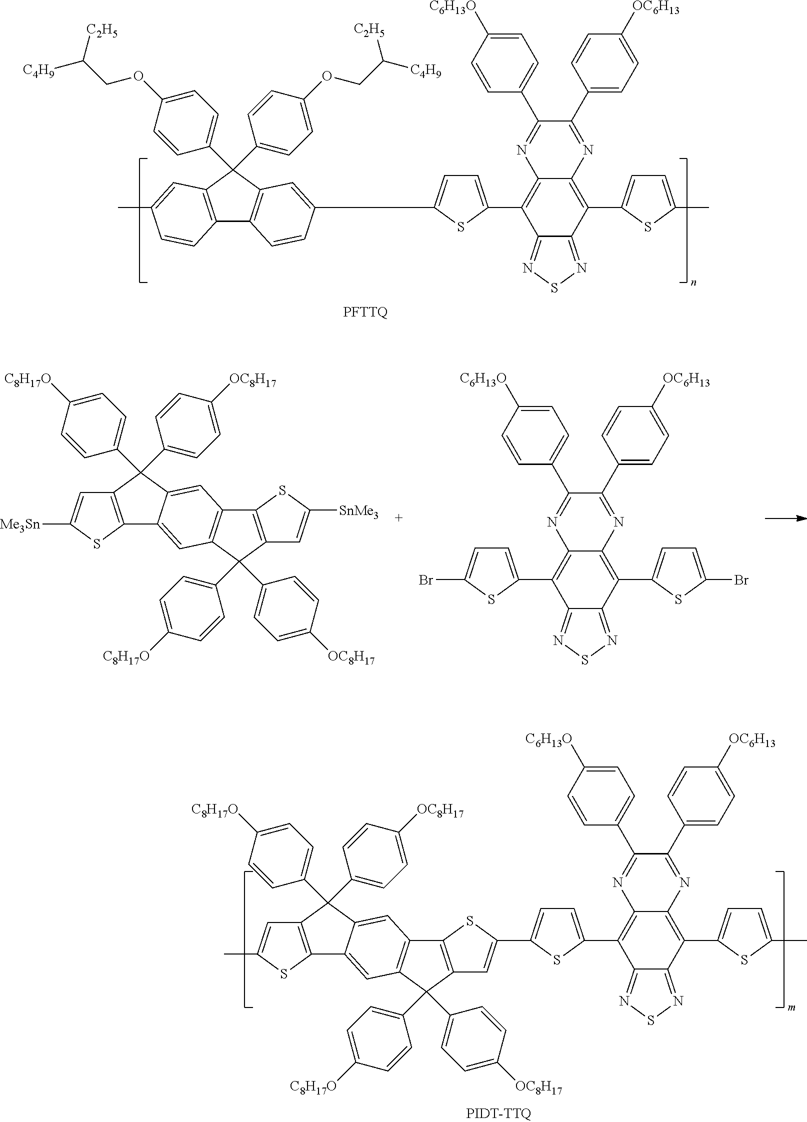

In Scheme 3, n for CP1 is preferably an integer from 3 to 100. n for PFTTQ is preferably an integer from 4 to 100. For PIDT-TTQ, m is preferably an integer from 4 to 100.

After synthesis of the conjugated polymers by cross coupling, conjugated polymer nanoparticles are generated. In one embodiment of the invention, the conjugated polymer that is dissolved in organic solvent is added to an aqueous solution, enabling the self-assembly of conjugated polymers into nanoparticles. In some embodiments of the invention, the organic solvent utilized in this step is selected from chloroform, methylene chloride, dimethylformamide, tetrahydrofuran, or dimethylsulfoxide. In preferred embodiments of the invention, the solvent is tetrahydrofuran.

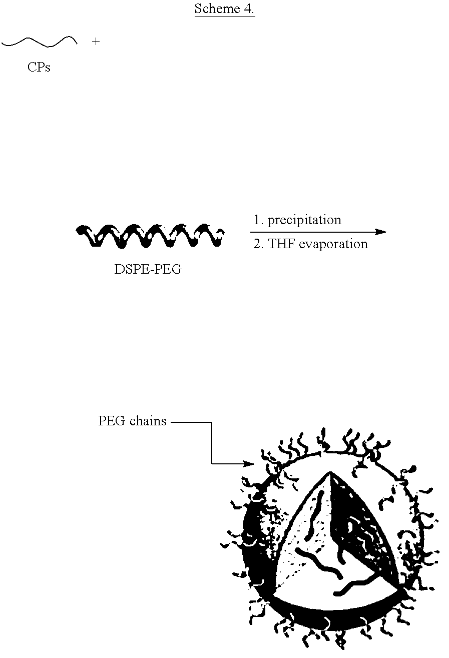

In another aspect of the invention, the conjugated polymers of the present invention are encapsulated into DSPE-PEG (1,2-Distearoyl-sn-Glycero-3-Phosphoethanolamine conjugated to polyethylene glycol) matrices to form conjugated polymer encapsulated nanoparticles by a traditional precipitation method (Scheme 4). Typically, a solution of conjugated polymer, for example P1 or PFTTQ, and DSPE-PEG in tetrahydrofuran is poured into 90% (v/v) water/THF solution. This is followed by sonication of the mixture for about 60 seconds using a microtip probe sonicator at about 12 W output. Without being bound to theory, the hydrophobic DSPE segments entangle with hydrophobic conjugated polymer molecules and the hydrophilic PEG chains extend into aqueous phase under sonication. The emulsion is then stirred at about room temperature overnight to evaporate the tetrahydrofuran, affording the conjugated polymer nanoparticles. In other aspects of the invention, the preceding method also applies to encapsulation into DSPE-PEG matrices functionalized by folic acid or another bioconjugate, as discussed in Example 5. In alternate embodiments of the invention, the nanoparticle is encapsulated into biocompatible matrices such as bovine serum albumin (BSA) protein, poly(lactic-co-glycolic acid) (PLGA) block copolymers, collagens or lipids.

In another embodiment of the invention, the conjugated polymer nanoparticles of the invention are used as sensors in biological applications, and have fluorescence properties that can be manipulated. In certain aspects of the invention, CP NPs particularly useful as fluorescence sensors in bioimaging are compounds of structure P1 in Scheme 2.

One major drawback of conventional microscopes is that diffraction of light reduces the spatial resolution achievable by a conventional microscope, which cannot distinguish between molecular or nanoscale probes that reside in close proximity to one another. However, the conjugated polymer nanoparticles of the invention are designed to have controllable fluorescence properties of the backbone, with “on” and “off” states. The control of “on” or “off” fluorescent states over time provides better tracking of specific biological species in vivo and can identify false positive signals at sub-diffraction scales. Representative conjugated polymer nanoparticles that fall within this embodiment of the invention are shown in Scheme 5.

In Scheme 5, the compound 1 was introduced into the side chains of P1 via click reaction to afford P2. As the structure of compound 1 can be reversibly converted induced by illumination with ultraviolet or visible light and the ring-closed isomer of compound 1 can quench the fluorescence of conjugated backbone, this unique property can be employed to control the fluorescence properties of conjugated backbone with “on” and “off” states. Scheme 5 gives the synthetic route to polymers P2 and P3. Compounds having “on” and “off” fluorescent states are not limited to example compounds P2 and P3 in Scheme 5. The invention encompasses a variety of reactive compounds with fluorescent structures, including compounds that can undergo electrocyclization reactions or intramolecular cyclization.

In Scheme 5, m and n are integers that indicate the number of repeat units in the conjugated polymer, as well as the relative ratio of the constituent functional groups. In certain aspects of the invention, m and n are each integers independently ranging from 0 to 100. In preferred aspects of the invention, m and n are each integers independently ranging from 0 to 50, and in more preferred aspects of the invention, m and n are each integers independently ranging from 0 to 20. Furthermore, the conjugated polymers of the invention are random copolymers.

In certain aspects of the invention, polymers with controllable fluorescence properties, such as P2, are used in the detection of cancer cells, or are used to detect tumors due to their surface functionalization with target ligands. In another aspect of the invention, the polymers are used in imaging or sensing applications with cells, tissues, or animals.

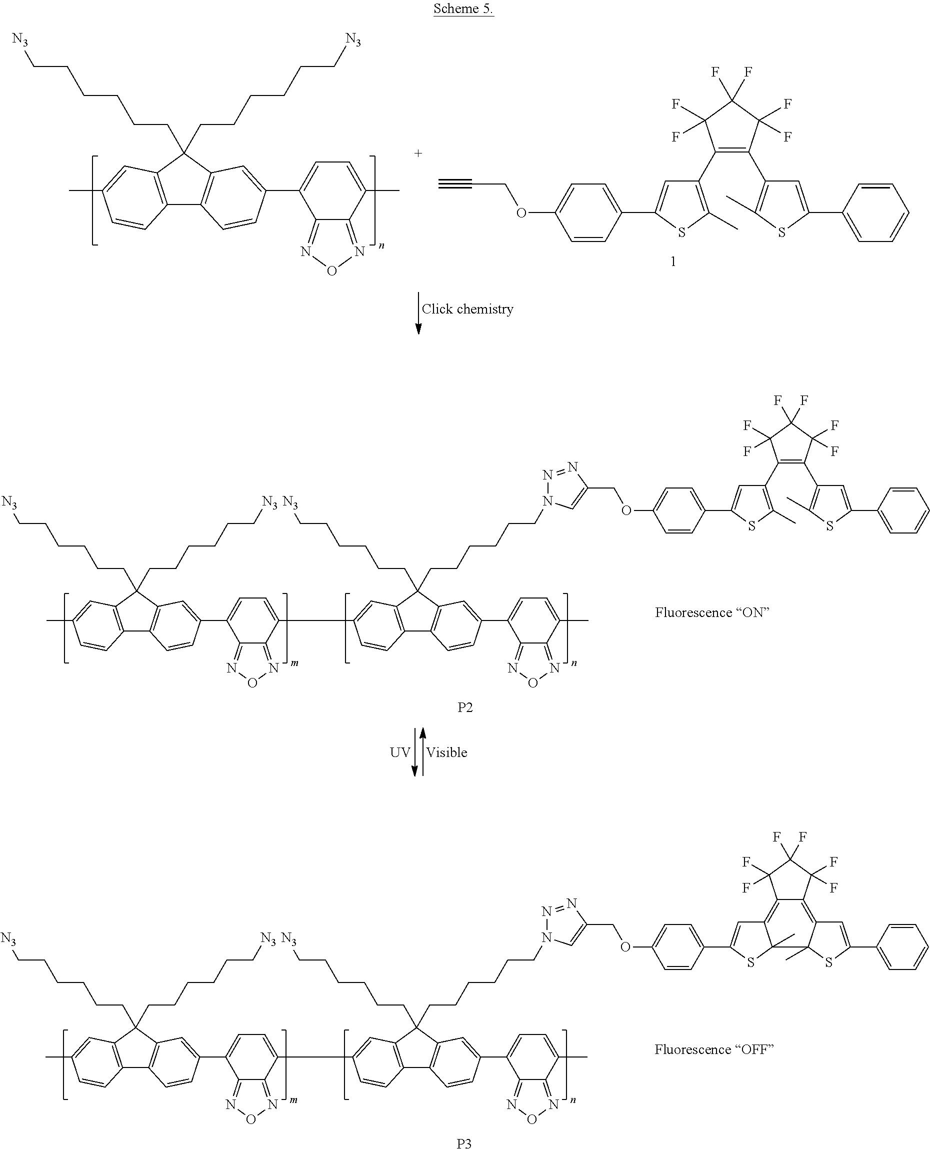

In another aspect of the invention, the conjugated polymer nanoparticles described herein may also be utilized in photodynamic therapy as photosensitizers. Scheme 6 gives some examples of molecules designed for photodynamic therapy. The photosensitizer group is incorporated onto the CP side chains through click chemistry. Energy transfer from the CP backbone to porphyrin acceptor can enhance the ability of porphyrin to generate reactive oxygen species, thus enable polymer amplified therapeutic effect in photodynamic therapy. In addition, some of these polymers by themselves can be used as photosensitizers for photodynamic therapy applications. The use of CP NPs in photodynamic therapy is not limited to compounds conjugated to porphyrin. CP NPs bearing groups such as phthalocyanines or molecules generating single oxygen species can be utilized as photosensitizers in photodynamic therapy.

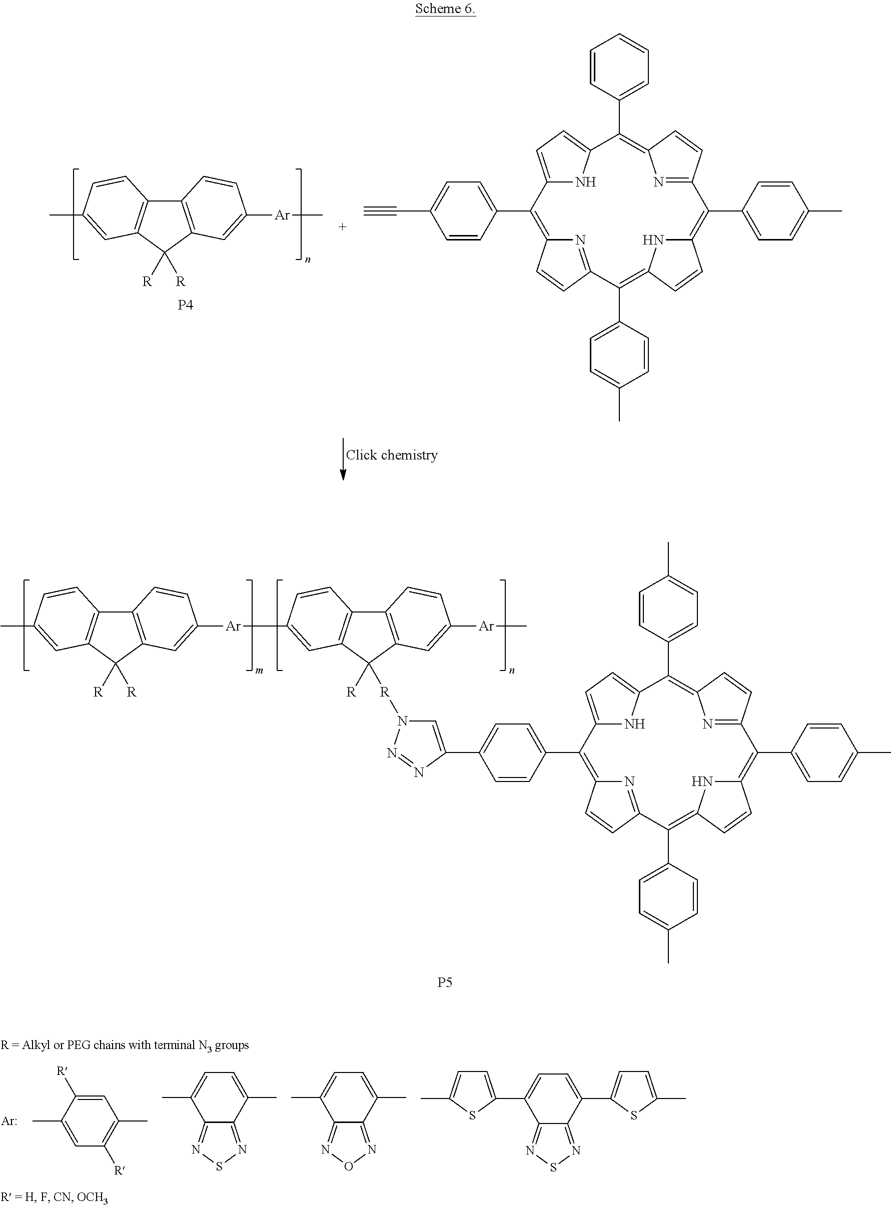

In Scheme 6, m and n are integers that indicate the number of repeat units in the conjugated polymer, as well as the relative ratio of the constituent functional groups. In certain aspects of the invention, m and n are each integers independently ranging from 0 to 100. In preferred aspects of the invention, m and n are each integers independently ranging from 0 to 50, and in more preferred aspects of the invention, m and n are each integers independently ranging from 0 to 20. Furthermore, the conjugated polymers of the invention are random copolymers.

Reaction of an alkyne with an azide as in Schemes 5 and 6 occurs by click chemistry. In some aspects of the invention, this reaction proceeds by catalysis with CuSO₄ and sodium ascorbate in tetrahydrofuran or dimethylsulfoxide.

In another aspect of the invention, the conjugated polymer backbone is affixed to hydrophilic side chains. The CP-based nanoparticles self-assemble in water. Scheme 7 gives examples of such CP-based nanoparticles. In particular, Scheme 7 shows the chemical structures of three NIR fluorescent conjugated polymers, which were used as representative polymers to illustrate the properties of the highly emissive FR/NIR fluorescent conjugated polymer-based nanoparticles revealed in this patent. The polymers poly[fluorene-co-di(thiophen-2-yl)-2,1,3-benzothiadiazole], poly[fluorene-co-2,1,3-benzothiadiazole-co-di(thiophen-2-yl)-2,1,3-benzothiadiazole] and poly[fluorene-co-2,1,3-benzoxadiazole-co-di(thiophen-2-yl)-2,1,3-benzothiadiazole] were denoted to PFDBT10-PEG1000-COOH, PFBTDBT10-PEG1000-COOH and PFBDDBT10-PEG1000-COOH, respectively.

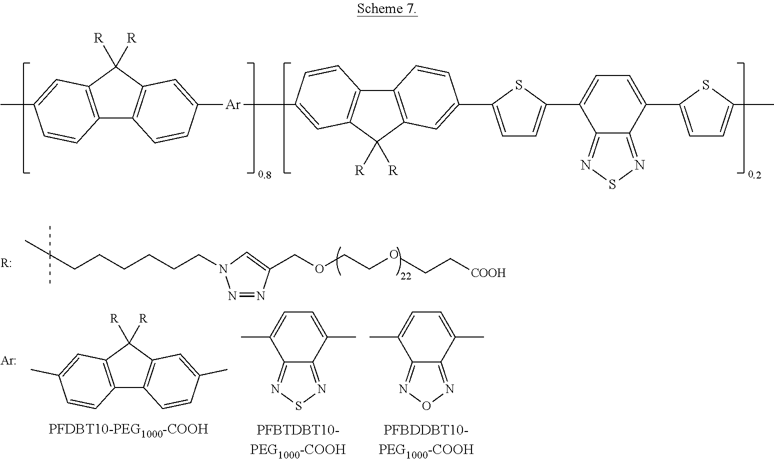

Another aspect of the invention relates to small molecular weight conjugated molecules. Scheme 8 depicts a synthetic route to such compounds, which are non-polymeric conjugated systems of carbocyclic and heterocyclic aromatic rings and carbon-carbon double bonds. Small molecular weight conjugated molecules are generated through a cross coupling of an organoborate reagent and a dihalide.

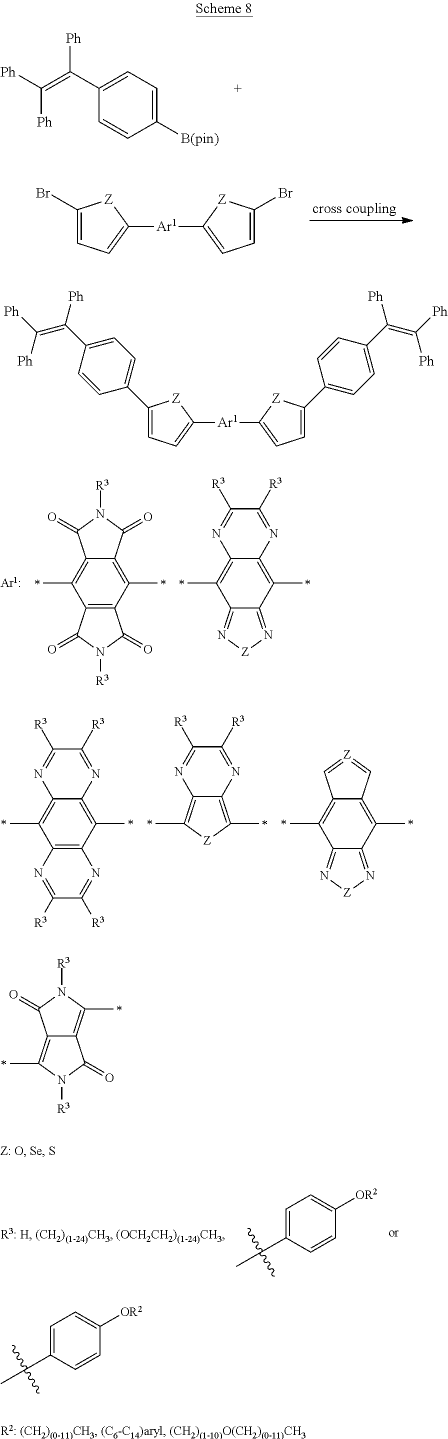

The organoborate reagent is a boronic acid (—B(OH)₂), a boronic acid ester such as a dimethyl borate (—B(OCH₃)₂), boronic acid pinacol ester (above, —B(pin):

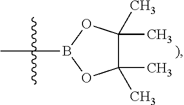

boronic acid catechol ester (—B(cat):

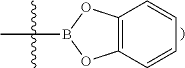 [SMILES: *B1Oc2ccccc2O1]

or a trifluoroborate (—BF₃K). In alternate embodiments of the invention, the organoborate reagent is instead an organostannane reagent such an a trialkylstannane reagent (e.g. —SnBu₃). Preferably the organoborate reagent is the diphenylethylene boronic acid pinacol ester pictured in Scheme 8. The dihalide of the invention is a polycyclic dihalide containing heterocyclic and optionally carbocyclic aromatic rings. The halogen is chloride, bromide or iodine. Preferably the halogen is bromide. The conditions for cross coupling are typical Suzuki or Stille cross coupling conditions well-known to those of ordinary skill in the art, and further described in the Examples section herein. Catalysts for the reaction include, but are not limited to from about 0.5 to about 10 mol % Pd₂(dba)₃ (wherein dba is dibenzylidene acetone), Pd(PPh₃)₄, NiCl₂, PdCl₂, or Ni(cod)₂ (wherein cod is cyclooctadiene) or other Pd(0), Pd(II), Ni(0) or Ni(II) catalysts. In preferred embodiments of the invention, the small molecular weight conjugated compound is the structure depicted in Scheme 8, wherein Ar¹ is:

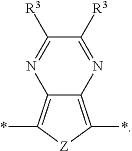

In some embodiments of the invention, the small molecular weight compounds in Scheme 8 are functionalized in order to increase specificity for an imaging target. For example, the alkyl, PEG, hydroxyphenyl or phenyl substituents of R³ can be functionalized with a fluorophore, a peptide, an oligonucleotide, a protein, and small molecule ligands through coupling chemistry, for example peptide coupling chemistry, or click chemistry, described herein for the conjugated polymer nanoparticles. The small molecular weight conjugated compounds of Scheme 8 are used in photoacoustic imaging of biological targets, such as tissues, brains, and live animals.

Another aspect of the present invention relates to enhancing the sensitivity of photoacoustic imaging through synthetic design. By fine-tuning the structure of the photoacoustic contrast reagent, the absorption region can be controlled (for example, to the NIR region) to avoid light scattering interference (for example, from the visible light region).

Conjugated polymers in general have not been implemented as contrast reagents in photoacoustic imaging applications. The present invention also relates to the use of the conjugated polymers described herein as contrast agents for photoacoustic imaging. An alternate embodiment of the invention relates to small molecular weight conjugated molecules also described herein as contrast reagents.

To ensure deep tissue penetration, the absorption of the CPs was fine-tuned to near-infrared (NIR) region. To facilitate their application in biological system, the CPs were formulated to be CP NPs by a traditional method using 1,2-distearoyl-sn-glycero-3-phosphoethanolamine-N-[methoxy(polyethylene glycol)-2000] as the matrix as described herein. The synthesized CP NPs demonstrated high PA intensities which are better than that of polyhydroxyfullerene (PHF) and commercial dye indocyanine green (ICG), which are currently widely used PA contrast agents. Preferred CP NPs of the present invention to use in photoacoustic imaging applications include PFTTQ and PIDT-TTQ.

The present invention also provides a method for photoacoustic imaging of a target, comprising:
incubating a target with a polymer of Formula (I) to form an incubated mixture;

irradiating the mixture with a pulsed laser, wherein the pulsed laser optically excites the polymer, to generate thermally-induced acoustic waves, wherein the acoustic waves result from energy emission from the excited polymer;
detecting the thermally-induced acoustic waves with ultrasound; and

translating the acoustic waves detected by ultrasound into an image of the target.

In addition, the synthesized CP NPs generate heat upon NIR light absorption. Therefore, another aspect of the invention relates to the use of the CP NPs described herein as a therapeutic agent for use in photothermal therapy. The present invention further provides a method for photothermal ablation of a cancer cell, comprising:

incubating a cancer cell with a conjugated polymer nanoparticle of formula (I) to form an incubated mixture; and

irradiating the mixture with a laser, wherein the polymer absorbs energy from laser irradiation and converts the energy to heat, thereby causing ablation of a cancer cell.

In some embodiments of the invention, the laser used in photothermal therapy is a pulsed laser.

The synthesized CPs based agents described herein provide a new platform for photothermal therapeutic applications and theranostic applications in which simultaneous diagnostic and therapeutic methods are administered.

### DEFINITIONS

All definitions set forth herein apply to standalone terms as well as when used as a component of a larger substituent (e.g. the definition of alkyl refers to alkyl and also the alkyl component of (C₁-C₁₀)alkoxy(C₁-C₆)alkyl.

“Alkyl” means a saturated aliphatic branched or straight-chain monovalent hydrocarbon radical. “(C₁-C₆) alkyl” means a radical having from 1-6 carbon atoms in a linear or branched arrangement. “(C₁-C₆)alkyl” includes methyl, ethyl, propyl, isopropyl, butyl, isobutyl, sec-butyl, pentyl, and hexyl.

“Alkylene” means a saturated aliphatic straight-chain divalent hydrocarbon radical having the specified number of carbon atoms. Thus, “(C₁-C₆)alkylene” means a divalent saturated aliphatic radical having from 1-6 carbon atoms in a linear arrangement. “(C₁-C₆)alkylene” includes methylene, ethylene, propylene, butylene, pentylene and hexylene.

As used herein, “aryl” or “aromatic” used alone or as part of a larger moiety includes both carbocyclic aromatic ring systems and heteroaromatic ring systems. These include monocyclic and polycyclic aromatic groups. The term “(C₆-C₁₄)aryl” used alone or as part of a larger moiety as in “arylalkyl”, “arylalkoxy”, or “aryloxyalkyl”, means carbocyclic aromatic rings. The term “carbocyclic aromatic group” may be used interchangeably with the terms “aryl”, “aryl ring” “carbocyclic aromatic ring”, “aryl group” and “carbocyclic aromatic group”. An aryl group typically has 6-14 ring atoms. A “substituted aryl group” is substituted at any one or more substitutable ring atom. The term “C₆₋₁₄ aryl” as used herein means a monocyclic, bicyclic or tricyclic carbocyclic ring system containing from 6 to 14 carbon atoms and includes phenyl, naphthyl, anthracenyl, 1,2-dihydronaphthyl, 1,2,3,4-tetrahydronaphthyl, fluorenyl, indanyl, indenyl and the like.

The term “heteroaryl”, “heteroaromatic”, “heteroaryl ring”, “heteroaryl group” and “heteroaromatic group”, used alone or as part of a larger moiety as in “heteroarylalkyl” or “heteroarylalkoxy”, refers to aromatic ring groups having five to fourteen ring atoms selected from carbon and at least one (typically 1-4, more typically 1 or 2).heteroatoms (e.g., oxygen, nitrogen, selenium or sulfur). They include monocyclic rings and polycyclic rings in which a monocyclic heteroaromatic ring is fused to one or more other carbocyclic aromatic or heteroaromatic rings. Heteroaromatic groups include, but are not limited to furan, oxazole, thiophene, 1,2,3-triazole, 1,2,4-triazine; 1,2,4-triazole, 1,2,5-thiadiazole 1,1-dioxide, 1,2,5-thiadiazole 1-oxide, 1,2,5-thiadiazole, 1,3,4-oxadiazole, 1,3,4-thiadiazole, 1,3,5-triazine, imidazole, isothiazole, isoxazole, pyrazole, pyridazine, pyridine, pyridine-N-oxide, pyrazine, pyrimidine, pyrrole, tetrazole, and thiazole. The term “5-14 membered heteroaryl” as used herein means a monocyclic, bicyclic or tricyclic ring system containing one or two aromatic rings and from 5 to 14 total ring atoms of which, unless otherwise specified, one, two, three, four or five are heteroatoms independently selected from N, NH, N(C₁₋₆alkyl), O and S.

Each aryl and heteroaryl is optionally and independently substituted. Exemplary substituents include halogen, (C₁-C₃)alkoxy, (C₁-C₃)alkylthio, hydroxy, (C₆-C₁₄)aryl, (C₅-C₁₄)heteroaryl, (C₃-C₁₅)cycloalkyl, (C₃-C₁₅)heterocyclyl, amino, (C₁-C₅)alkylamino, (C₁-C₅)dialkylamino, thio, oxo, (C₁-C₅)alkyl, (C₅-C₁₄)aryl(C₁-C₅)alkyl, (C₅-C₁₄)heteroaryl(C₁-C₅)alkyl, nitro, cyano, sulfonato, phosphonato, carboxylate, hydroxyl(C₁-C₅)alkyl and halo(C₁-C₅)alkyl. “Tolyl” as used herein means a phenyl group substituted by a methyl group, at either the ortho, meta or para position.

The term “alkoxy” means —O-alkyl, wherein alkyl is defined above; “hydroxyalkyl” means alkyl substituted with hydroxy; “aryl alkyl” means alkyl substituted with an aryl group; “alkoxyalkyl” mean alkyl substituted with an alkoxy group; “alkylamine” means amine substituted with an alkyl group; “cycloalkylalkyl” means alkyl substituted with cycloalkyl; “dialkylamine” means amine substituted with two alkyl groups. Thus, “(C₁-C₆)alkoxy(C₁-C₁₂)alkyl” means an alkyl group having from 1-12 carbon atoms in a linear or branched arrangement, that is additionally substituted at any one of carbons 1-12 by an alkoxy group having from 1-6 carbon atoms in a linear or branched arrangement. The organic fragment to which the (C₁-C₆)alkoxy(C₁-C₁₂)alkyl is attached may be bonded at any one of carbon atoms 1-12 on the (C₁-C₁₂)alkyl chain.

“Organoborate” as used herein is a reactive boron species suitable for use as a cross-coupling reaction partner. “Organoborate” includes boronic acids of the formula R—B(OH)₂, organoboronic esters of the formula R—B(pin) or R—B(cat), or organotrifluoroborates of the formula R—BF₃K, where R is the organic fragment to which the boron atom is attached. In R—B(pin), “(pin)” stands for pinacol, wherein the oxygen atoms of the pinacol diol are covalently bound to boron. In R—B(cat), “cat” stands for catechol, wherein the oxygen atoms of the catechol diol are covalently bound to boron.

A “biocompatible matrix” is a scaffold that supports a chemical compound or a polymer that serves to perform an appropriate function in a specific application without causing an inappropriate or undesirable effect in a host system. Examples of biocompatible matrices include poly(ethylene glycol), 1,2-Distearoyl-sn-glycero-3-phosphoethanolamine-N-[methoxy(polyethylene glycol)](DSPE-PEG), poly(DL-lactide-co-glycolide), chitosan, bovine serum albumin, and gelatin. In a matrix, and particularly in a DSPE-PEG matrix, PEG can mean, for example, PEG1000 (averaging a molecular weight of 1000), PEG2000 ((averaging a molecular weight of 2000), PEG5000 (averaging a molecular weight of 5000). As a side chain on a conjugated polymer, PEG means a polyethylene glycol polymer made up of 1 to 100 repeat units, or more preferably 1-50 repeat units.

“Folate” means a folic acid derivative, often a salt, which is covalently bonded to a molecule, for example a conjugated polymer. Generally, the folate or folic acid salt is covalently bonded through the terminal primary carboxylic acid.

A “bioconjugate” as used herein is typically a small molecule with an affinity for a biological target, for example a complex in a cell membrane.

“Ablation” means loss of cellular function through death of a cell.

“Theranostic” means simultaneous diagnostic and therapeutic method.

### EXAMPLES

The following examples are provided to illustrate-one or more preferred embodiments of the invention, but are not limited embodiments thereof. Numerous variations can be made to the following examples that lie within the scope of the invention.

### Example 1

### Representative Synthetic Procedure for Low Molecular Weight Conjugated Molecules

To a solution of 4,9-bis(5-bromothiophen-2-yl)-6,7-bis(4-(hexyloxy)phenyl)-[1,2,5]thiadiazolo[3,4-g]quinoxaline (86.0 mg, 0.1 mmol), 1-(4-(4,4,5,5-tetramethyl-1,3,2-dioxaborolan-2-yl)phenyl)-1,2,2-triphenylethylene (114.5 mg, 0.25 mmol), Pd(PPh₃)₄ (11.5 mg, 0.01 mmol) and tetrabutylammonium bromide (32.2 mg, 0.01 mmol) in toluene (15 mL) was added K₂CO₃ aqueous solution (2 M, 5 mL). The reaction was performed at 90° C. for 24 h under argon atmosphere. After removing the solvent, the residua was purified through silica gel column chromatography using hexane/dichloromethane (7/3) as eluent to afford 6,7-bis(4-(hexyloxy)phenyl)-4,9-bis(5-(4-(1,2,2-triphenylvinyl)phenyl)thiophen-2-yl)-[1,2,5]thiadiazolo[3,4-g]quinoxaline as black solid (98.2 mg, yield: 72%). Each compound shown in Scheme 1 was made by the analogous procedure.

### Example 2

### Synthesis of Conjugated Polymer CP-1

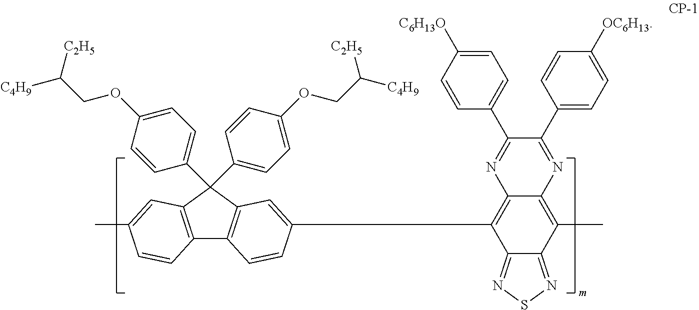

A Schlenk tube was charged with 4,9-dibromo-6,7-bis(4-(hexyloxy)phenyl)-[1,2,5]thiadiazolo[3,4-g]quinoxaline (139.6 mg, 0.20 mmol), 2,7-bis(4,4,5,5-tetramethyl-1,3,3-dioxaboralan-2-yl)-9,9-bis(4-(2-ethylhexyloxyl)phenyl)fluorene (165.2 mg, 0.20 mmol), palladium acetate (4 mg, 0.018 mmol) and tricyclohexylphosphine (10 mg, 0.036 mmol) in toluene (10 mL) before it was sealed with a rubber septum. The Schlenk tube was degassed with three freeze-pump-thaw cycles to remove air. After the mixture was heated to 80° C., an aqueous Et₄NOH solution (20 wt %, 1.5 mL) was added to initiate the reaction. After 3 days, the reaction was stopped and cooled down to room temperature. The mixture was dropped slowly into methanol (100 mL) to precipitate the crude polymer followed by centrifugation. The crude polymer was subsequently redissolved in chloroform (200 mL), washed with water 3 times, and dried over MgSO₄. After solvent removal, the polymer (68 mg, yield: 31%) was obtained as a black solid by precipitation in methanol. ¹H NMR (500 MHz, CDCl₃, ppm) δ: 8.09 (br, 4H), 7.44-7.23 (br, 10H), 6.72 (br, 8H), 3.94 (br, 4H), 3.69 (br, 4H), 1.76 (br, 4H), 1.63 (br, 2H), 1.43-1.25 (br, 28H) 0.89-0.83 (br, 18H).

### Example 3

### Synthesis of PFTTQ

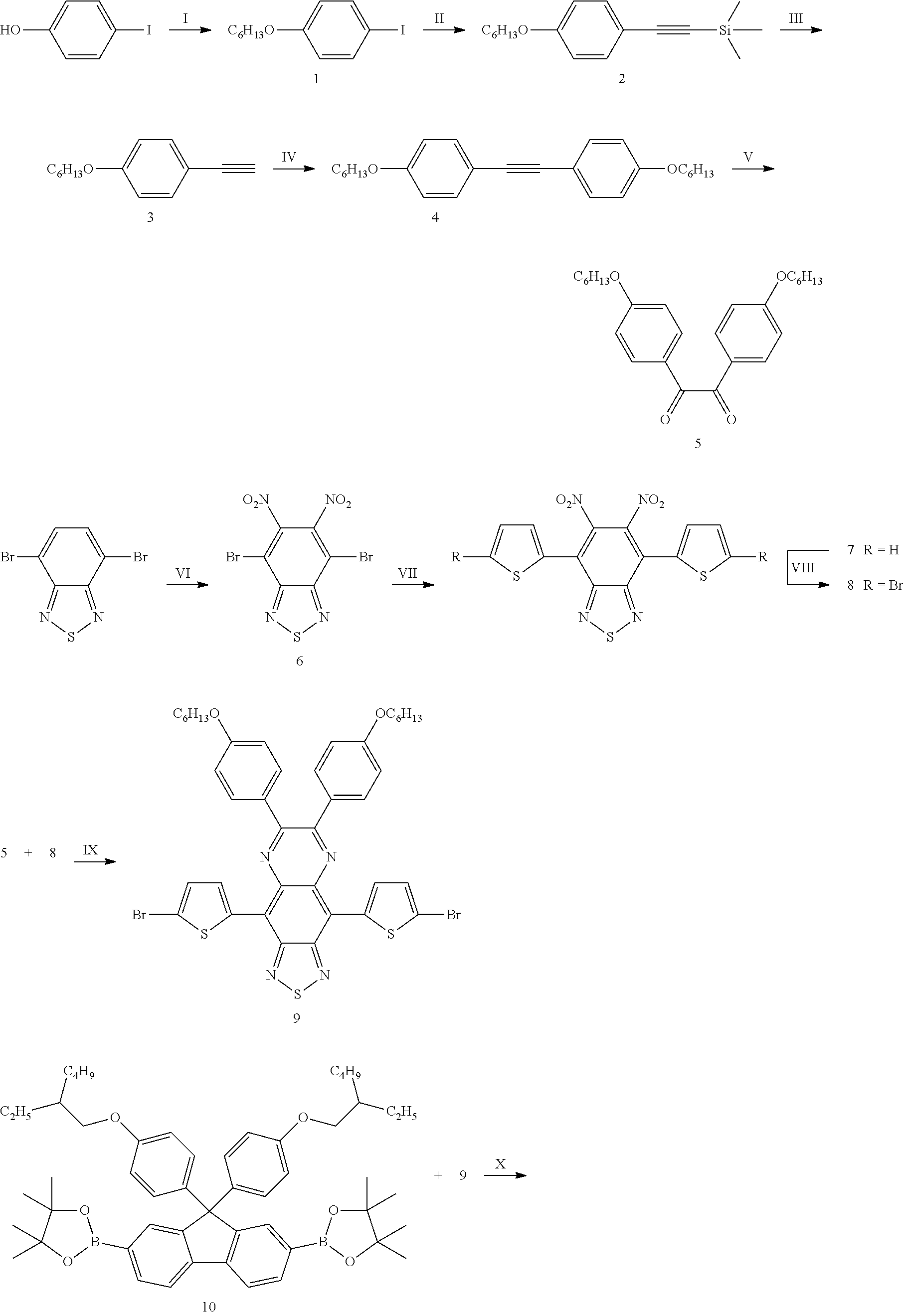

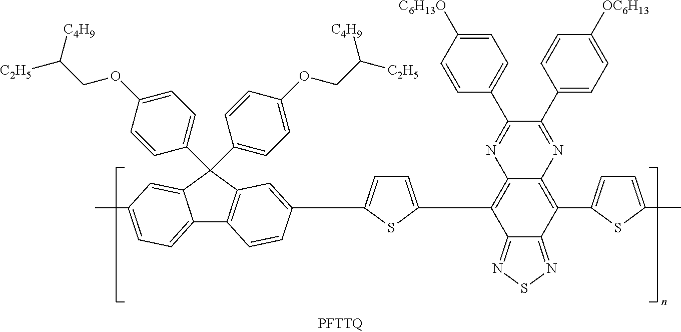

The preceding synthetic route to PFTTQ is detailed in the procedures below. Reagents and conditions: i) KOH, 1-bromohexane, H₂O, 100° C., 1 h; ii) CuI, Pd(PPh₃)₂Cl₂, trimethylsilylaceylene, i-Pr₂NH/THF, room temperature, overnight; iii) KOH, THF/MeOH/H₂O, room temperature, 1 h; iv) CuI, Pd(PPh₃)₂Cl₂, 1, i-Pr₂NH/THF, room temperature, overnight; v) KMnO₄, NaHCO₃, TBAB, CH₂Cl₂, room temperature, 2 days; vi) H₂SO₄, HNO₃, 100° C., overnight; vii) Pd(PPh₃)₂Cl₂, 2-(tributylstannyl)thiophene, THF, 80° C., overnight; viii) NBS, DMF, 60° C., 3 h; ix) a) Iron, acetic acid; b) acetic acid, 135° C., 24 h; x) Pd(OAc)₂, Cy₃P, Et₄NOH, toluene, 18 h.

### 1-Hexyloxy-4-iodobenzene (1)

To a solution of tetrabutylammonium bromide (100 mg, 0.31 mmol) and 4-iodophenol (5 g, 22.7 mmol) in aqueous potassium hydroxide (50 mL, 50 wt %) at 100° C. was added 1-bromohexane (4.37 mL, 31.1 mmol) in one portion. The reaction was kept for 1 h at 100° C. before it was cooled to room temperature. The mixture was extracted with dichloromethane, washed with water and dried over MgSO₄. After solvent removal, the residue was purified by silica gel column chromatography using hexane as eluent to provide 1 as a colorless liquid (6.3 g, yield: 91%). ¹H NMR (500 MHz, CDCl₃, ppm) δ: 7.53 (d, J=8.5 Hz, 2H), 6.67 (d, J=8.5 Hz, 2H), 3.91 (t, J=6.5 Hz, 2H), 1.75 (m, 2H), 1.45 (m, 2H), 1.33 (m, 4H), 0.91 (t, J=7 Hz, 3H). ¹³C NMR (125 MHz, CDCl₃, ppm) δ: 159.06, 138.16, 116.97, 82.40, 68.16, 31.57, 29.14, 25.69, 22.60, 14.04.

### (4-(Hexyloxy)phenyl)ethynyl)trimethylsilane (2

To a solution of 1-hexyloxy-4-iodobenzene (3.25 g, 10.69 mmol), copper iodide (104 mg, 0.54 mmol) and Pd(PPh₃)₂Cl₂ (150 mg, 0.21 mmol) in diisopropylamine/tetrahydrofuran (30/10 mL) at room temperature under argon atmosphere was added trimethylsilylaceylene (2.45 g, 25 mmol) via syringe. The reaction was performed at room temperature overnight. The mixture was diluted with dichloromethane, filtered through a celite pad, concentrated under reduced pressure and purified by silica gel column chromatography using hexane as eluent to afford 2 as a colorless liquid (2.8 g, yield: 95%). ¹H NMR (500 MHz, CDCl₃, ppm) δ: 7.38 (d, J=8.5 Hz, 2H), 6.79 (d, J=8.5 Hz, 2H), 3.94 (t, J=6.5 Hz, 2H), 1.75 (m, 2H), 1.45 (m, 2H), 1.33 (m, 4H), 0.89 (t, J=7 Hz, 3H), 0.29 (s, 9H). ¹³C NMR (125 MHz, CDCl₃, ppm) δ: 159.37, 133.44, 115.02, 114.36, 105.34, 92.27, 68.07, 31.57, 29.15, 25.68, 22.58, 14.00, 0.08.

### 1-Ethynyl-4-(hexyloxy)benzene (3)

A round bottle flask was charged with ((4-(hexyloxy)phenyl)ethynyl)trimethylsilane (2.74 g, 10 mmol), potassium hydroxide (5.6 g, 100 mmol), THF (50 mL), methanol (25 mL) and water (18 mL). The mixture was stirred at room temperature under argon atmosphere for 1 h. After solvent removal, the residue was subsequently redissolved in dichloromethane, washed with water and dried over MgSO₄. The crude product was purified by silica gel column chromatography using hexane as eluent to yield 3 as a colorless liquid (1.9 g, yield: 94%). ¹H NMR (500 MHz, CDCl₃, ppm) δ: 7.42 (d, J=8.5 Hz, 2H), 6.83 (d, J=8.5 Hz, 2H), 3.95 (t, J=6.5 Hz, 2H), 3.00 (s, 1H), 1.77 (m, 2H), 1.46 (m, 2H), 1.35 (m, 4H), 0.93 (t, J=7 Hz, 3H). ¹³C NMR (125 MHz, CDCl₃, ppm) δ: 159.59, 133.57, 114.49, 113.95, 83.80, 75.67, 68.08, 31.59, 29.17, 25.71, 22.61, 14.03.

### 1,2-Bis(4-(hexyloxy)phenyl)ethyne (4)

A solution of 1-ethynyl-4-(hexyloxy)benzene (2.02 g, 10 mmol), 1-hexyloxy-4-iodobenzene (3.19 g, 10.5 mmol), copper iodide (95 mg, 0.50 mmol) and Pd(PPh₃)₂Cl₂ (140 mg, 0.20 mmol) in diisopropylamine (30 mL) was stirred for 20 h at room temperature under argon atmosphere. After solvent removal, the residue was subsequently redissolved in dichloromethane, washed with water and dried over MgSO₄. The crude product was purified by silica gel column chromatography (hexane/dichloromethane=9/1) to afford 4 as a white solid (2.6 g, yield: 69%). ¹H NMR (500 MHz, CDCl₃, ppm) δ: 7.42 (d, J=8.5 Hz, 4H), 6.84 (d, J=8.5 Hz, 4H), 3.97 (t, J=6.5 Hz, 4H), 1.78 (m, 4H), 1.46 (m, 4H), 1.35 (m, 4H), 0.91 (t, J=7 Hz, 6H). ¹³C NMR (125 MHz, CDCl₃, ppm) δ: 158.99, 132.83, 115.54, 114.52, 87.96, 68.09, 31.59, 29.19, 25.71, 22.59, 14.02.

### 1,2-Bis(4-(hexyloxy)phenyl)ethane-1,2-dione (5)

To a solution of 1,2-bis(4-(hexyloxy)phenyl)ethyne (2.5 g, 6.6 mmol) in dichloromethane (20 mL) was added tetrabutylammonium bromide (100 mg, 0.31 mmol), NaHCO₃ (1.2 g, 14.3 mmol), KMnO₄ (3.3 g, 20.9 mmol) in water (40 mL). The mixture was vigorously stirred at room temperature for 2 days, and sodium bisulfite (8 g) and hydrochloric acid (10 mL) were subsequently added. The mixture was extracted with dichloromethane and washed with water, and the organic phase was dried over MgSO₄. After solvent removal, the residue was purified by silica gel column chromatography (hexane/ethyl acetate=19/1) to afford 5 as a white solid (2.5 g, yield: 92%). ¹H NMR (500 MHz, CDCl₃, ppm) δ: 7.92 (d, J=8.5 Hz, 4H), 6.94 (d, J=8.5 Hz, 4H), 4.03 (t, J=7 Hz, 4H), 1.80 (m, 4H), 1.46 (m, 4H), 1.34 (m, 4H), 0.91 (t, J=7 Hz, 6H). ¹³C NMR (125 MHz, CDCl₃, ppm) δ: 193.56, 164.50, 132.35, 126.13, 114.72, 68.48, 31.50, 28.98, 25.60, 22.56, 13.99.

### 4,7-Dibromo-5,6-dinitrobenzo[1,2,5]thiodiazole (6)

To a mixture of sulphuric acid (10 mL) and nitric acid (70%, 10 mL) at 0° C. was added 4,7-dibromo-2,1,3-benzothiodiazole (2 g, 6.8 mmol). After stirring at 100° C. overnight, the mixture was cooled down to room temperature and poured into ice-water (100 mL), which was followed by addition of sodium hydroxide solution to neutralize the excess acid. The precipitate was filtered and washed with water. The crude product was purified by silica gel column chromatography (hexane/dichloromethane=8/2) to afford 6 as a white solid (522 mg, yield: 20%). MS: m/z=383.9.

### 5,6-Dinitro-4,7-di(thiophen-2-yl)benzo[1,2,5]thiadiazole (7)

A solution of 4,7-dibromo-5,6-dinitro-benzothiadiazole (250 mg, 0.65 mmol), 2-(tributylstannyl)thiophene (971 mg, 2.6 mmol) and dichlorobis(triphenyl-phosphine)palladium (45 mg, 0.65 mmol) in anhydrous THF was heated to 80° C. under argon atmosphere. The mixture was allowed to react at 80° C. overnight. After the solvent removal, the residue was purified by silica gel column chromatography (hexane followed by hexane/dichloromethane=7:3) to afford 7 as an orange solid (230 mg, yield: 90.7%). ¹H NMR (500 MHz, CDCl₃, ppm) δ: 7.78 (d, J=4 Hz, 2H), 7.55 (d, J=4 Hz, 2H), 7.27 (t, J=4 Hz, 2H).

### 4,7-Bis(5-bromothiophen-2-yl)-5,6-dinitrobenzo[1,2,5]thiadiazole (8)

A solution of 5,6-dinitro-4,7-di(thiophen-2-yl)benzo[1,2,5]thiadiazole (230 mg, 0.59 mmol) in DMF (20 mL) was heated to 60° C. N-bromosuccinimide (215 mg, 1.21 mmol) was added in one portion to the mixture. After 1 h, another portion of NBS (215 mg, 1.21 mmol) was added, and the reaction was continued for additional 1 h. The reaction mixture was cooled down to room temperature and poured into water. The collected precipitation was washed with methanol twice to afford 8 as an orange solid (274 mg, yield: 85%). ¹H NMR (500 MHz, CDCl₃, ppm) δ: 7.29 (d, J=4 Hz, 2H), 7.22 (d, J=4 Hz, 2H).

### 4,9-Bis(5-bromothiophen-2-yl)-6,7-bis(4-(hexyloxy)phenyl)-[1,2,5]thiadiazolo[3,4-g]quinoxaline (9)

A mixture of 4,7-bis(5-bromothiophen-2-yl)-5,6-dinitrobenzo[1,2,5]thiadiazole (300 mg, 0.55 mmol) and fine iron powder (368 mg, 6.5 mmol) in acetic acid (20 mL) was heated to 80° C. After 6 h, the mixture was cooled down to room temperature and filtered off. The filtration was mixed with 1,2-bis(4-(hexyloxy)phenyl)ethane-1,2-dione (225 mg, 0.55 mmol) and stirred at 135° C. under argon atmosphere for 24 h. After solvent removal, the residue was washed with methanol. The precipitation was purified by silica gel column chromatography (hexane/dichloromethane=7/3) to afford 9 as a black-green solid (241 mg, yield: 51%). ¹H NMR (500 MHz, CDCl₃, ppm) δ: 8.83 (d, J=4 Hz, 2H), 7.70 (d, J=8.5 Hz, 4H), 7.21 (d, J=4 Hz, 2H), 6.96 (d, J=8.5 Hz, 4H), 4.05 (t, J=6.5 Hz, 4H), 1.85 (m, 4H), 1.54 (m, 4H), 1.38 (m, 8H), 0.93 (t, J=7 Hz, 6H). ¹³C NMR (125 MHz, CDCl₃, ppm) δ: 160.63, 153.01, 150.67, 137.32, 133.79, 133.05, 132.50, 129.94, 129.40, 120.21, 119.46, 114.11, 68.19, 31.68, 29.30, 25.81, 22.66, 14.09.

### Poly[9,9-bis(4-(2-ethylhexyl)phenyl)fluorene-alt-co-6,7-bis(4-(hexyloxy)phenyl)-4,9-di(thiophen-2-yl)thiadiazoloquinoxaline](PFTTQ)

A Schlenk tube was charged with 4,9-Bis(5-bromothiophen-2-yl)-6,7-bis(4-(hexyloxy)phenyl)-[1,2,5]thiadiazolo[3,4-g]quinoxaline (100.0 mg, 0.116 mmol), 2,7-bis(4,4,5,5-tetramethyl-1,3,3-dioxaboralan-2-yl)-9,9-bis(4-(2-ethylhexyloxyl)phenyl)fluorene (95.8 mg, 0.116 mmol), palladium acetate (3 mg) and tricyclohexylphosphine (7 mg) in toluene (10 mL) before it was sealed with a rubber septum. The Schlenk tube was degassed with three freeze-pump-thaw cycles to remove air. After the mixture was heated to 80° C., an aqueous Et₄NOH solution (20 wt %, 1.5 mL) was added to initiate the reaction. After 18 h, the reaction was stopped and cooled down to room temperature. The mixture was dropped slowly into methanol (100 mL) to precipitate the crude polymer followed by centrifugation. The crude polymer was subsequently redissolved in chloroform (200 mL), washed with water 3 times, and dried over MgSO₄. After solvent removal, the polymer (45 mg, yield: 30%) was obtained as a black-green solid by precipitation in methanol. ¹H NMR (500 MHz, CDCl₃, ppm) δ: 9.00 (br, 4H), 7.81-7.14 (br, 14H), 6.80 (br, 8H), 3.98 (br, 4H), 3.78 (br, 4H), 1.83 (br, 4H), 1.67 (br, 2H), 1.50-1.26 (br, 28H), 0.93-0.85 (br, 18H).

### Example 4

### Synthesis of PIDT-TTQ

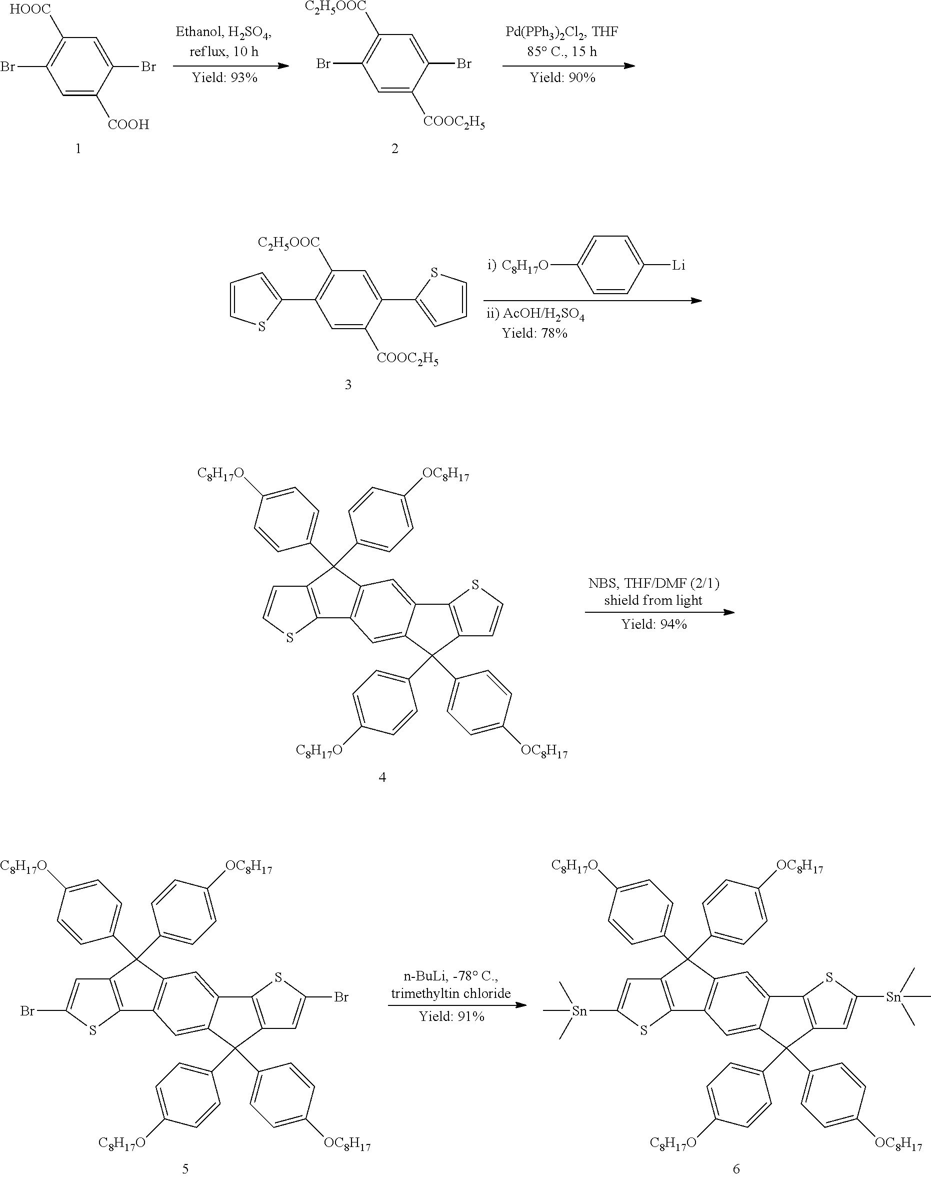

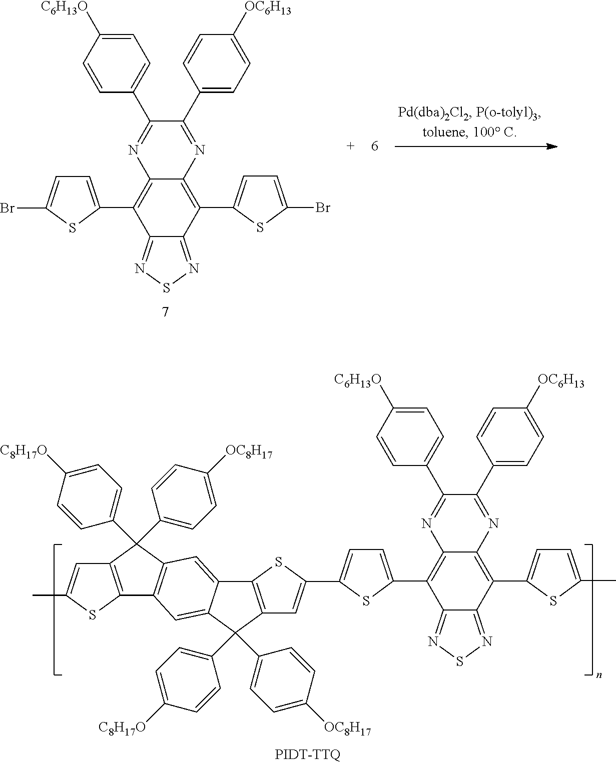

### Diethyl 2,5-dibromoterephalate (2)

To a solution of 2,5-dibromoterephthalic acid (10.0 g, 30.8 mmol) in absolute ethanol (50 mL) was added concentrated H₂SO₄ (15 mL). The mixture was refluxed overnight. After cooling to room temperature, the precipitate was collected by filtration and then further purified by recrystallization from ethanol to give diethyl 2,5-dibromoterephalate as a white crystal. Yield: 93%.

### Diethyl 2,5-di(thien-2-yl)terephthalate (3)

To a solution of diethyl 2,5-dibromoterephthalate (4.3 g, 11.3 mmol) and Pd(PPh₃)₂Cl₂ (397.1 mg, 565.7 μmol) in anhydrous THF (15 mL) was added 2-(tributylstannyl)thiophene (10.8 mL, 33.9 mmol). The reaction mixture was stirred at 85° C. under argon atmosphere for 15 h. After cooled to room temperature, the mixture was treated with KF aqueous solution (10 wt %, 50 mL) for 2 h. The product was extracted with dichloromethane three times, and the combined organic phase was washed with water three times and then dried over MgSO₄. After filtration, the solvent was removed under reduced pressure. The residue was purified by silica gel chromatography (hexane/ethyl acetate=9/1) to afford diethyl 2,5-di(thien-2-yl)terephthalate as a white solid. Yield: 90%. ¹H NMR (400 MHz, CDCl₃, ppm) δ: 7.83 (s, 2H), 7.41 (dd, 2H), 7.12 (m, 4H), 4.24 (q, 4H), 1.18 (t, 6H).

### 4,4,9,9-Tetrakis(4-(octyloxy)phenyl)-4,9-dihydro-s-indaceno[1,2-b:5,6-b]dithiophene (4)

A solution of 1-bromo-4-octyloxybenzene (9.975 g, 35.0 mmol) in anhydrous THF (70 mL) was treated with n-BuLi (21.87 mL, 1.6 M in hexane, 35 mmol) at −78° C. for 1 h. Then a solution of diethyl 2,5-di(thien-2-yl)terephthalate (1.93 g, 5.0 mmol) in anhydrous THF (10 mL) was added slowly into the reaction mixture. After addition completed, the reaction mixture was stirred at −78° C. for another 1 h and slowly warmed to room temperature for another 12. The solution was quenched with water (20 mL). After solvent removal, the residue was subsequently extracted with dichloromethane (100 mL×3), washed with water (100 mL×3) and dried over MgSO₄. After filtration and solvent removal, the crude product was dissolved in boiling acetic acid (100 mL) and dropwisely added with 3 drops of concentrated H₂SO₄. The reaction was allowed to reflux for 3 h and then quenched with water (100 mL). After solvent removal, the residue was subsequently extracted with dichloromethane (100 mL×3), washed with water (100 mL×3) and dried over MgSO4. The crude product was purified by silica gel column chromatography using hexane/dichloromethane (8/2) as eluent to yield 4,4,9,9-Tetrakis(4-(octyloxy)phenyl)-4,9-dihydro-s-indaceno[1,2-b:5,6-b]dithiophene as light yellow solid. Yield: 78%.

### 2,7-Dibromo-4,4,9,9-tetrakis(4-(octyloxy)phenyl)-4,9-dihydro-s-indaceno[1,2-b:5,6-b]dithiophene (5)

To a solution of 4,4,9,9-tetrakis(4-(octyloxy)phenyl)-4,9-dihydro-s-indaceno[1,2-b:5,6-b]dithiophene (3.50 g, 3.23 mmol) in THF/DMF (2/1, 75 mL) was added N-bromosuccinimide (1.26 g, 7.11 mmol) in portions within 20 min. This mixture was stirred for 3 h in the presence of light at room temperature and then the solvent was removed under reduced pressure. The residue was subsequently dissolved in dichloromethane (200 mL), washed with water (3×100 mL) and dried over MgSO₄. After concentration, the residue was purified by silica gel chromatography (hexane/dichloromethane=7/3) to afford 2,7-dibromo-4,4,9,9-tetrakis(4-(octyloxy)phenyl)-4,9-dihydro-s-indaceno[1,2-b:5,6-b]dithiophene as a white solid (3.80 mg, yield: 94%). ¹H NMR (500 MHz, CDCl₃, ppm) δ: 7.31 (s, 2H), 7.14 (d, J=8.5 Hz, 8H), 6.98 (s, 2H), 6.80 (d, J=8.5 Hz, 8H), 3.92 (t, J=6 Hz, 8H), 1.76 (m, 8H), 1.44 (m, 8H), 1.32 (m, 32H), 0.90 (t, 12H). ¹³C NMR (150 MHz, CDCl₃, ppm) δ: 158.65, 155.61, 153.35, 141.57, 136.30, 135.33, 129.32, 126.38, 117.47, 114.78, 114.19, 68.41, 63.18, 32.21, 29.74, 29.63, 26.48, 23.04, 14.47.

### (4,4,9,9-Tetrakis(4-(octyloxy)phenyl)-4,9-dihydro-s-indacenol[1,2-b:5,6-b]dithiophene-2,7-diyl)bis(trimethylstannane) (6)

To a solution of 2,7-dibromo-4,4,9,9-tetrakis(4-(octyloxy)phenyl)-4,9-dihydro-s-indaceno[1,2-b:5,6-b]dithiophene (1.8 g, 1.45 mmol) in anhydrous THF (50 mL) at −78° C. was added n-butyllithium (1.6 M, 2.17 mL, 3.48 mmol) dropwise over 10 min. The reaction mixture was stirred at −78° C. for 1 h, and then trimethyltin chloride (1 M in hexane, 4.0 mL, 4.00 mmol) was added in one portion. The reaction was allowed to warm to room temperature and stirred overnight. The mixture was concentrated and subsequently redissolved in diethyl ether (250 mL), washed with water and dried over MgSO₄. Evaporation of the solvent followed by recrystallization from methanol afforded (4,4,9,9-tetrakis(4-(octyloxy)phenyl)-4,9-dihydro-s-indacenol[1,2-b:5,6-b]dithiophene-2,7-diyl)bis(trimethylstannane) as a light brownish solid. (1.87 g, Yield: 91%)

### Poly[(4,4,9,9-tetrakis(4-(octyloxy)phenyl)-4,9-dihydro-s-indacenol[1,2-b:5,6-b]dithiophene-2,7-diyl)-alt-co-4,9-bis(thiophen-2-yl)-6,7-bis(4-(hexyloxy)phenyl)-[1,2,5]thiadiazolo[3,4-g]quinoxaline](PIDT-TTQ)

A Schlenk tube was charged with (4,4,9,9-tetrakis(4-(octyloxy)phenyl)-4,9-dihydro-s-indacenol[1,2-b:5,6-b]dithiophene-2,7-diyl)bis(trimethylstannane) (164 mg, 0.116 mmol), 4,9-bis(5-bromothiophen-2-yl)-6,7-bis(4-(hexyloxy)phenyl)-[1,2,5]thiadiazolo[3,4-g]quinoxaline (100 mg, 0.116 mmol), Pd₂(dba)₃ (5.3 mg, 5.8 μmol) and P(o-tolyl)₃ (7.1 mg, 23.2 μmol) and toluene (12 mL). The Schlenk tube was degassed with argon by three freeze-pump-thaw cycles to remove air. The mixture was heated to 100° C. and stirred for 40 h. After cooling down to room temperature, the mixture was dropped slowly into methanol (100 mL). The precipitate was collected by centrifugation. Then the crude polymer was subsequently redissolved in dichloromethane (200 mL), washed with water by three times, and dried over MgSO₄. After filtration, the mixture was concentrated to ˜5 mL and added dropwise into methanol to precipitate the polymer. The polymer was collected by centrifugation and dried in vacuum oven to afford the polymer as solid. (101 mg, Yield: 49%).

### Example 2

### Self-Assembly of CP-Based Nanoparticles in Water

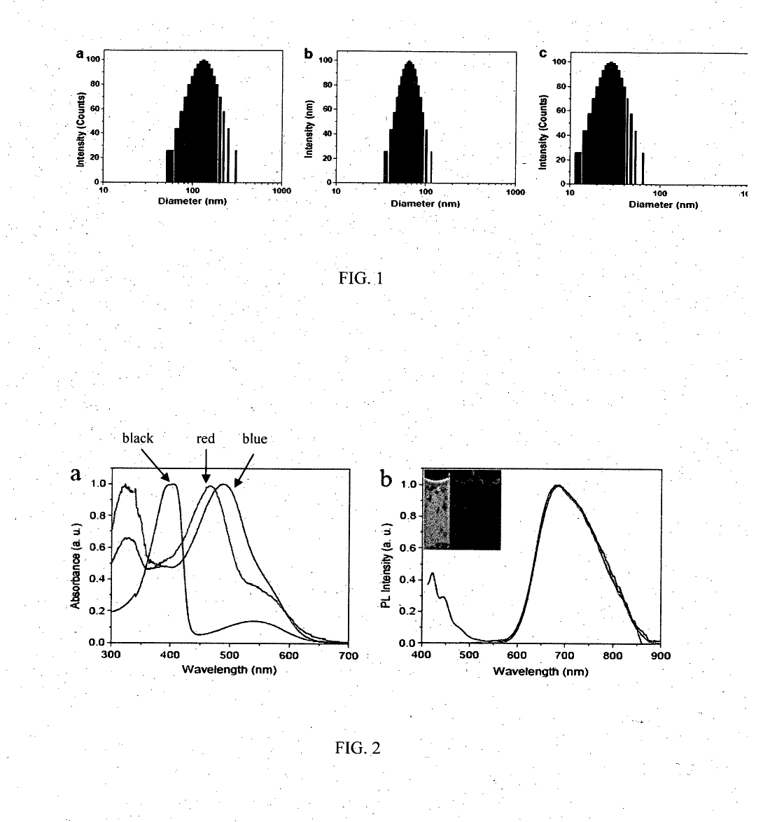

A set of PFBDDBT10-PEG1000-COOH NPs were prepared by adding 2 mL of DMSO containing CP with concentration of 0.5, 0.25 and 0.17 mg/mL into 10 mL of Milli-Q water, respectively. FIG. 1 shows the laser light scattering (LLS) results of PFBDDBT-PEG1000-COOH in water with different CP feeding concentrations. As shown, the particle size decreases from 116 nm to 28 nm with decreasing the CP concentration in DMSO, indicating that the size of CP NPs can be controlled by fabrication procedures.

### Example 3

### Spectroscopy of CP-Based Nanoparticles in Water

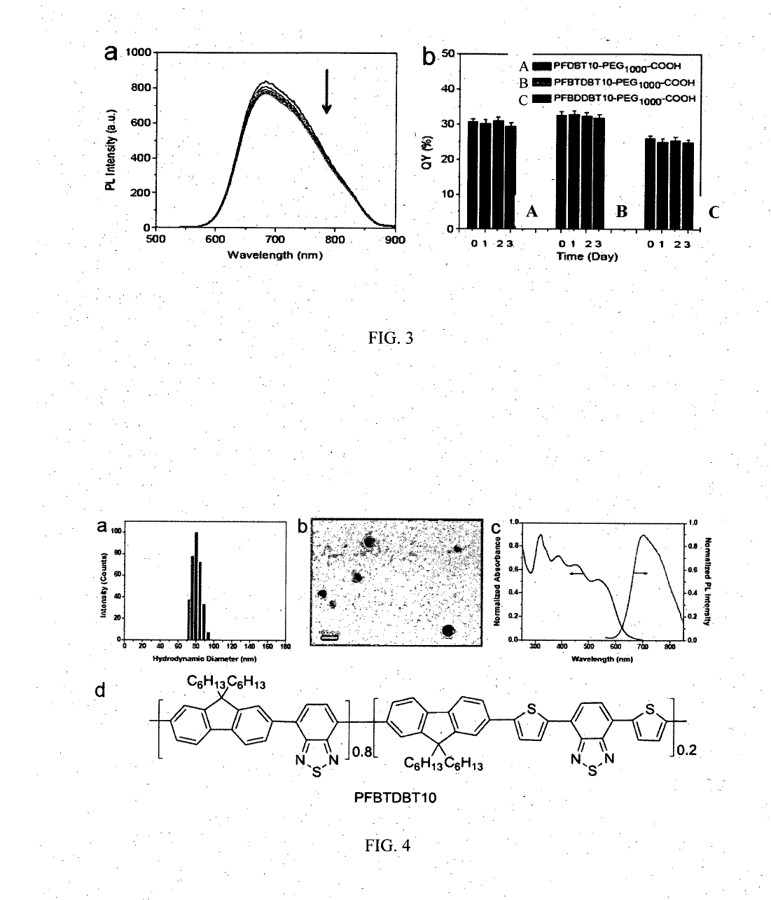

FIG. 2 shows the UV-vis and PL spectra of PFDBT-PEG1000-COOH, PFBTDBT-PEG1000-COOH and PFBDDBT-PEG1000-COOH NPs in water, respectively. Although the three CP NPs exhibit different absorption spectra, they show almost identical PL spectra centered at 680 nm. This is because that they have the same NBG2 unit of the vicinity of 4,7-di(thiophen-2-yl)-2,1,3-benzothiadiazole (DBT) units. The emission spectra extend very broad from 550 to 900 nm, and most are located in NIR region. Furthermore, the NPs have large Stokes shift from 192 to 277 nm, which minimizes the interference between the absorption and emission spectra. The PL spectra of the NPs match the confocal laser scanning microscope (CLSM) with 650 nm long-pass barrier filter for signal collection. The quantum yields of PFDBT-PEG1000-COOH, PFBTDBT-PEG1000-COOH and PFBDDBT-PEG1000-COOH in water were measured to be 30±1%, 32±1% and 25±1%, respectively, and 46±1%, 59±1% and 45±1% in DMSO, respectively, using 4-(dicyanomethylene)-2-methyl-6-(p-dimethylaminostyryl)-4H-pyran (DCM) in methanol as a standard (43%).[30] By far, these are the highest values for NIR fluorescent CP NPs in water. Compared to those in DMSO, the QYs of the CPEs in water only decrease by ˜33-45%, which is a much smaller drop in comparison with previous reported CPEs [29, 31], where the decrease even up to ten to hundred times. This indicates that the PEG₁₀₀₀ side chains provide a hydrophobic microenvironment for the conjugated backbone against water invasion, thus suppressing ICT-induced fluorescence quenching in water.

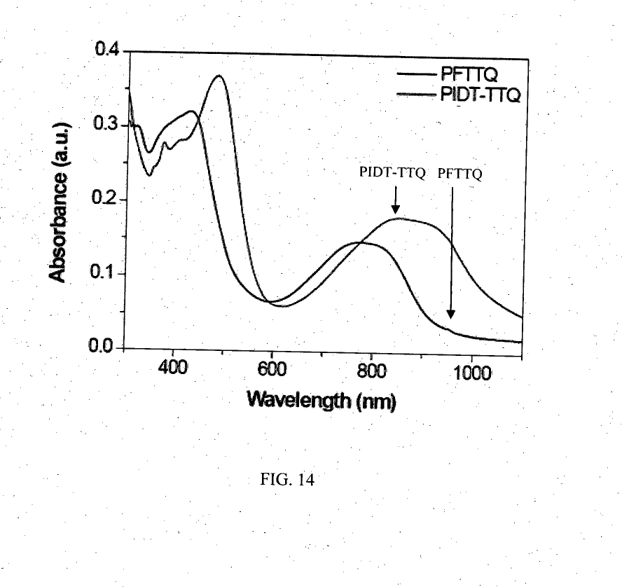

FIG. 11 shows the UV-vis and PL spectra of CP1 nanoparticles (NPs) in water. The absorption spectrum of the CP1 nanoparticles has two peaks centered at 455 and 563 nm, which correspond to π-π* transition of the conjugated backbone and charge transfer state, respectively. The NPs exhibits a PL spectrum with an emission peak at 685 nm. The quantum yield (QY) of the NPs based on CP1 was measured to be 7±1%, using rhodamine 6G in methanol as reference (QY=95%). FIG. 14 shows the absorption spectra of PFTTQ and PIDT-TTQ NPs in water. PFTTQ has two absorption peaks located at ˜430 and 775 nm, which are attributed to π-π* transition of the conjugated backbone and charge transfer state, respectively. PIDT-TTQ NPs show a broad UV-vis-NIR absorption band from 620 nm to 1100 nm. Both PFTTQ and PIDT-TTQ NPs have strong absorption at 800 nm, where the light has deep penetration in biological tissue.

### Example 4

### Photoluminescence Measurement in the Presence of Bovine Serum Albumin

Since the optical signals of CPEs used as cellular probes are often disturbed by nonspecific interactions between the probes and biomolecules, bovine serum albumin (BSA) was chosen as the model biomolecule to study the effect of nonspecific interactions on fluorescence due to its abundance in culture medium, and surfactant-like hydrophobic interactions with small fluorophores, and charged or neutral CPEs in aqueous media. FIG. 3a shows the representative PL spectra of PFBDDBT10-PEG1000-COOH in 150 mM PBS in the absence and presence of BSA with concentrations from 0 to 0.25 μM. As shown, only a slight decrease in fluorescence intensity of the CP NPs was observed upon increasing the BSA concentration with the saturation occurring at [BSA]=0.10 μM, indicating that small variation occurs for the local microenvironment of the conjugated backbones due to the protection of PEG chains.

To evaluate the thermal stability of the optical properties of the CP NPs, three CP NPs were incubated in PBS/BSA (150 mM/0.25 μM) mixture at 37° C. for 3 days, respectively. The variations of the quantum yields for PFDBT10-PEG1000-COOH, PFBTDBT10-PEG1000-COOH and PFBDDBT10-PEG1000-COOH are shown as a bar graph in FIG. 3b. During the 3-day experiments, no obvious changes in quantum yields were observed, indicating that these CP NPs have good thermal stability.

### Example 5

### Nanoparticle Assembly by Encapsulation into a Matrix

The folic acid (FA)-functionalized PFBTDBT10-loaded 1,2-distearoyl-sn-glycero-3-phosphoethanolamine-N-[methoxy(poly ethylene glycol)](DSPE-PEG) NPs were synthesized using a modified nanoprecipitation method.[32]A tetrahydrofuran (THF) solution containing CP, DSPE-PEG2000 and DSPE-PEG5000-FA (50 mol % of the matrix) was added into water under sonication, resulting in the hydrophobic DSPE segments entangling with hydrophobic CP molecules and hydrophilic PEG chains extending into the aqueous phase. After evaporation of THF, the CP-loaded DSPE-PEG-FA NP suspension was collected after further purification using a 0.2 μm syringe filter. As a control group, CP-encapsulated DSPE-PEG2000 NPs without surface folic acid functionalization were also fabricated following the same procedure. LLS results reveal that CP-loaded DSPE-PEG NPs with and without folate functionalization have similar volume average hydrodynamic diameters of ˜80 nm (FIG. 4a). High-resolution transmission electron microscopy (HR-TEM) observation was conducted to study the morphology of the CP-loaded DSPE-PEG-FA NPs. As shown in FIG. 4b, the NPs are nearly spherical in shape with an average size of ˜60 nm, which is smaller than LLS result due to the shrinkage in dry sample state in the HR-TEM observation. FIG. 4c shows the UV-vis absorption and PL spectra of CP-loaded DSPE-PEG-FA NP suspension in water. The NPs have four absorption maxima at 320, 383, 448 and 535 nm, respectively, and an emission peak centered at 698 nm, which is beneficial to bioimaging applications. Moreover, the quantum yield of CP-loaded DSPE-PEG-FA NPs in water is measured to be 21%, using 4-(dicyanomethylene)-2-methyl-6-(p-dimethylaminostyryl)-4H-pyran in methanol as a standard (43%).

The preceding procedure of encapsulating organic conjugated polymers into DSPE-PEG matrices is applicable to conjugated polymer nanoparticles with or without folic acid functionalization. Both PFTTQ NPs and PIDT-TTQ NPs were synthesized following the preceding procedure. Notably, no obvious precipitation was observed from the prepared nanoparticle solutions after storage at 4° C. for 3 months, indicating their excellent colloidal stability.

### Example 6

### Examination of Thermal Stability of CP-Based NPs

The thermal stability of the optical properties of CP-loaded DSPE-PEG-FA NPs was investigated upon incubation of the NPs in PBS buffer at 37° C. using organic dyes, e.g. Alexa Fluor 555 and Rhodamine 6G, as the controls. As shown in FIG. 5a, no decrease in fluorescence intensity of CP-loaded DSPE-PEG-FA NPs is observed after their incubation in PBS at 37° C. for 7 days. In comparisons, Alexa Fluor 555 and Rhodamine 6G show ˜18% and ˜25% decrease in PL intensity upon incubation in PBS at 37° C. for 7 days, respectively. This result suggests that CP-loaded DSPE-PEG-FA NPs has better thermal stability than the widely used organic dyes. In addition, CP-loaded DSPE-PEG-FA NPs show little hydrodynamic size change when incubation in PBS buffer at 37° C. for 7 days (FIG. 5b), indicating their high kinetic stability in aqueous environment.

### Example 7

### Application to Cancer Cell Imaging

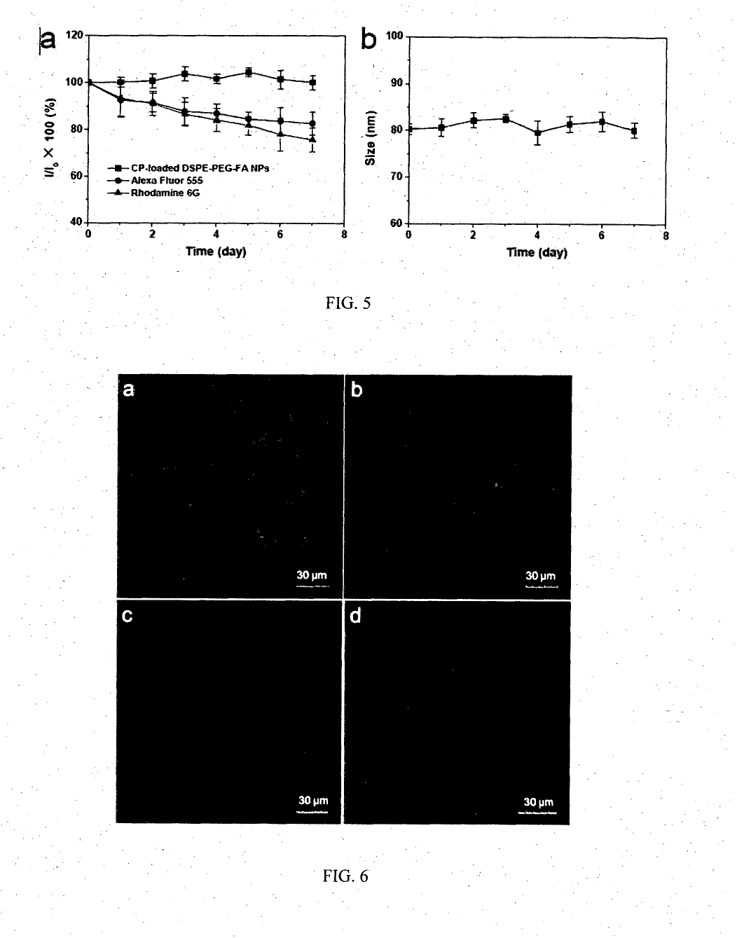

Cell imaging based on CP-loaded DSPE-PEG NPs with and without folate functionalization was investigated with confocal laser scanning microscopy (CLSM). MCF-7 breast cancer cells and NIH/3T3 fibroblast cells were used to demonstrate the utility of CP-loaded DSPE-PEG-FA NPs in targeted cancer cell imaging. The CLSM images of CP-loaded DSPE-PEG-FA NP-stained and CP-loaded DSPE-PEG NP-stained MCF-7 cells are shown in FIGS. 6a and 6b, respectively. Obvious red fluorescence can be observed for both the NP-stained MCF-7 cells, which are discretely localized in the cell cytoplasm. In addition, the fluorescence intensity in FIG. 6a is much higher than that in FIG. 6b, indicating that more FA-functionalized NPs are internalized into the MCF-7 cancer cells that overexpress folate receptors in the cell membrane through the receptor-mediated endocytosis. The specific targeting ability of CP-loaded DSPE-PEG-FA NPs to MCF-7 cancer cells were further assessed by NIH/3T3 fibroblast cells. As shown in FIGS. 6c and 6d, the fluorescence intensity of CP-loaded DSPE-PEG-FA NP-stained NIH/3T3 fibroblast cells is similar to that of CP-loaded DSPE-PEG NP-stained NIH/3T3 cells, which is because of the low expression of folate receptors in the NIH/3T3 cell membrane.

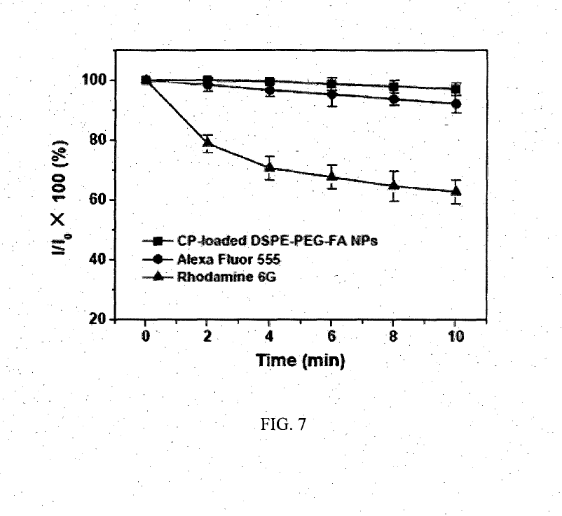

The photostability comparisons among CP-loaded DSPE-PEG-FA NPs, Alexa Fluor 555 and Rhodamine 6G in MCF-7 cancer cells were studied under continuous laser scanning upon excitation at 543 nm. As shown in FIG. 7, CP-loaded DSPE-PEG-FA NPs show ˜3% decrease in PL intensity after continuous laser excitation at 543 nm for 10 min, which has better photostability than those for Alexa Fluor 555 (˜8% decrease) and Rhodamine 6G (˜37% decrease).

Compared to PFBDDBT10-PEG1000-COOH, two new peaks centered at 280 and 320 nm for PFBDDBT10-PEG1000-FA ascribed to folic acid absorption appeared after conjugation, suggesting the success of the introduction of FA groups (FIG. 13), while the emission resembles with each other. The NPs have a mean diameter of 40 nm. The QY of PFBDDBT10-PEG₁₀₀₀-FA in water was determined to be 24±1% using DCM in methanol as a reference.

### Example 8

### Fluorescence Imaging in Mouse Model

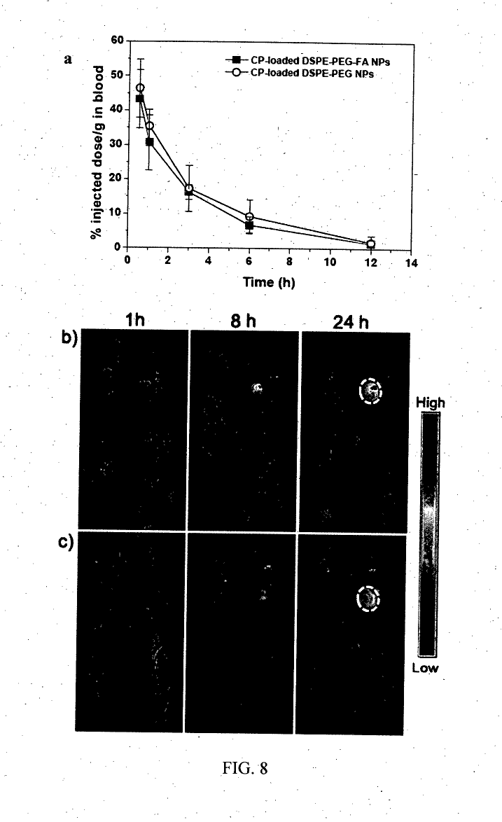

For the animal experiments, the animal model was established by subcutaneously inoculating murine hepatic H22 cancer cells with overexpressed folate receptors into the left axillary space of ICR mice. The CP-loaded NP levels in the blood over time after intravenous injection of NPs with and without folate functionalization, respectively, were investigated by determining the CP fluorescence in blood at various time points post injection (FIG. 8a). The in vivo blood circulation half-life values of CP-loaded DSPE-PEG NPs with and without folate functionalization are calculated to be ˜1.7 and ˜1.8 h, respectively. FIGS. 8b and 8c show the time-dependent biodistribution profile and tumor accumulation of CP-loaded DSPE-PEG NPs with and without folate in H22 tumor bearing mice, respectively, using a Maestro EX in vivo fluorescence imaging system. Although the NPs are widely dispersed among the whole body of mouse within 1 h post-injection, they tend to accumulate in the tumor tissue over time. Moreover, much higher fluorescence intensity is observed at the tumor site of folate-functionalized NP-treated mouse as compared to that of CP-loaded DSPE-PEG NP-treated mouse at all time points post-injection. This result suggests that CP-loaded DSPE-PEG-FA NPs are able to preferentially accumulate in tumor tissues not only through passive targeting resulting from the enhanced permeability and retention (EPR) effect [33] but also via folate receptor-mediated active targeting effect.

### Example 9

### Ex-Vivo Fluorescence Imaging

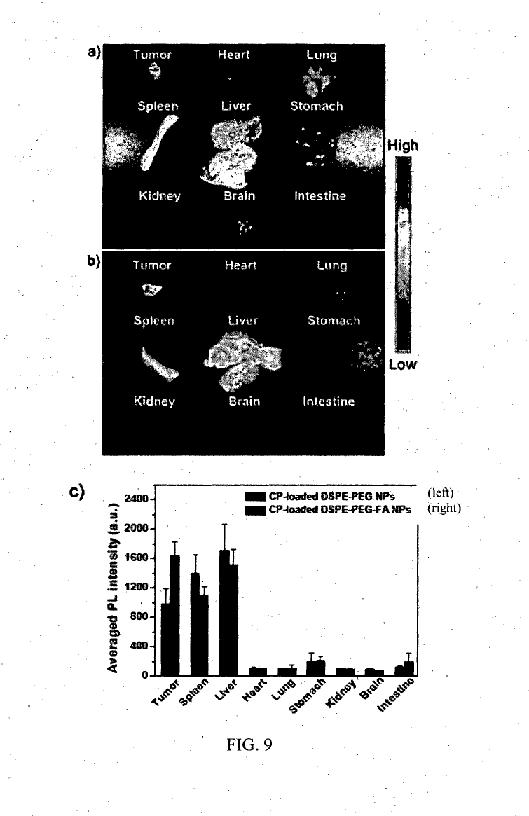

FIGS. 9a and 9b show the ex vivo fluorescence images of various organs at 24 h post intravenous injection of CP-loaded DSPE-PEG NPs with and without folate functionalization, respectively. The organs including heart, lung, spleen, liver, stomach, kidney, brain, intestine and tumor were isolated to assess the tissue distribution of the NPs. Fluorescence signals can be observed in tumor, liver and spleen tissues at 24 h post-injection, indicating that the NPs are mainly accumulated in these tissues. In addition, as shown in FIG. 9c, the average fluorescence intensity at the tumor site of folate-functionalized NP-treated mouse (1626.2±193.6) is ˜1.7 times higher than that of CP-loaded DSPE-PEG NP-treated mouse (981.0±202.6), confirming the active targeting ability of CP-loaded DSPE-PEG-FA NPs in in vivo studies.

### Example 10

### Toxicity Studies of CP-Based Nanoparticles in Mouse Model

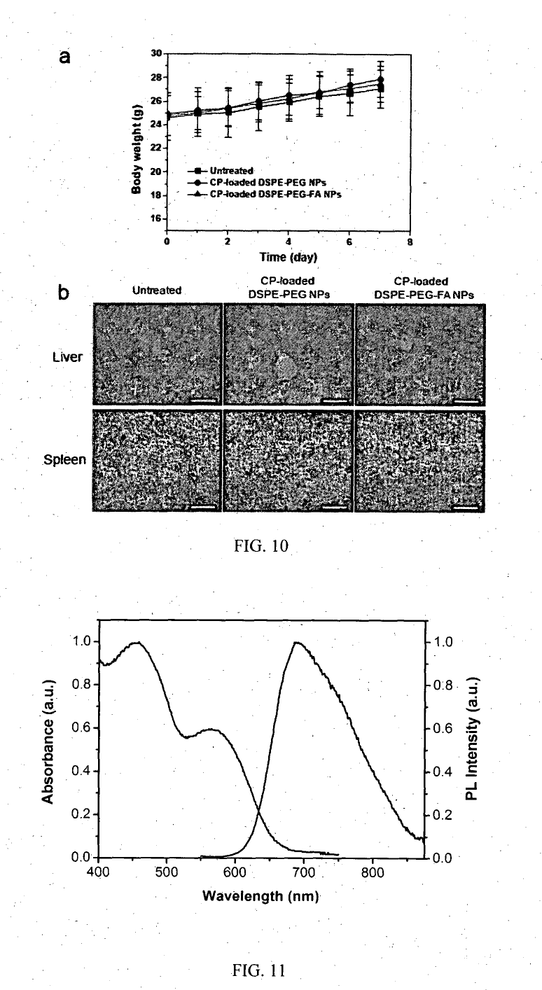

The in vivo toxicity of CP-loaded DSPE-PEG-FA NPs was studied using healthy tumor-free mice. As shown in FIG. 10a, neither mortality nor body weight loss of the mice post intravenous injection of CP-loaded DSPE-PEG NPs with and without folate functionalization, respectively, is observed as compared to the untreated and healthy mice. Histological analyses were also conducted to evaluate the in vivo toxicity of CP-loaded NPs. After 7 days post intravenous injection of CP-loaded DSPE-PEG NPs with and without folate functionalization, respectively, the mice were sacrificed and the liver as well as spleen tissues were excised, sliced, and performed H&E staining. The H&E-stained slices from the organs, were evaluated by 3 independent pathologists. FIG. 10b shows the representative images taken from the H&E-stained slices, which reveal that both the NP treatments do not cause any significant lesion to the tested organs. These results indicate that CP-loaded NPs have no obvious in vivo toxicity to the mice, which is essential for bioimaging applications.

### Example 11

### Toxicity in Cancer Cell Imaging

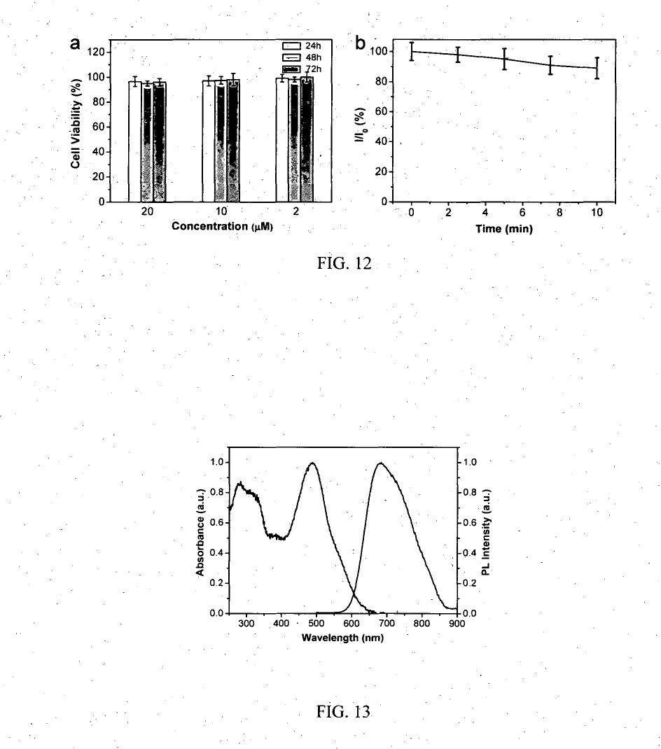

The cytotoxicity of PFBDDBT10-PEG₁₀₀₀-FA against MCF-7 breast cancer cells was evaluated by MTT assays. FIG. 12a shows the cell viability after incubation with the PFBDDBT10-PEG₁₀₀₀-FA with concentration of 2, 10, 20 μM (based on repeat unit) for 24, 48 and 72 h, respectively. It is worth noting that PFBDDBT10-PEG₁₀₀₀-FA shows low cytotoxicity even at 20 μM after 72 h, thus making it a safe bioimaging probe for targeted cancer cell imaging.

The photostability of PFBDDBT10-PEG₁₀₀₀-FA was investigated under continuous laser scanning upon excitation at 488 nm with 10% laser power (2.5 mW). As shown in FIG. 12b, after 10-minute continuous laser illumination, the fluorescence intensity from PFBDDBT10-PEG₁₀₀₀-FA stained MCF-7 cancer cells decreases ˜10%, which is obviously better than that for many commercial organic dyes.[34] This revealed that the PFBDDBT10-PEG₁₀₀₀-FA has high photostability, which is essential for bioimaging applications, especially in long-term studies.

### Example 12

### Photoacoustic (PA) Signal Measurement

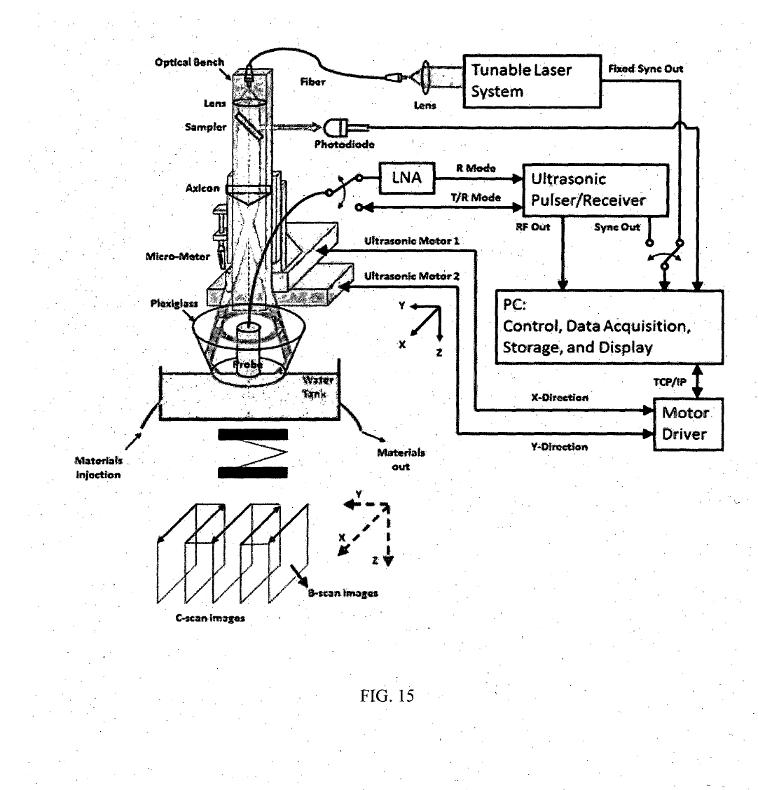

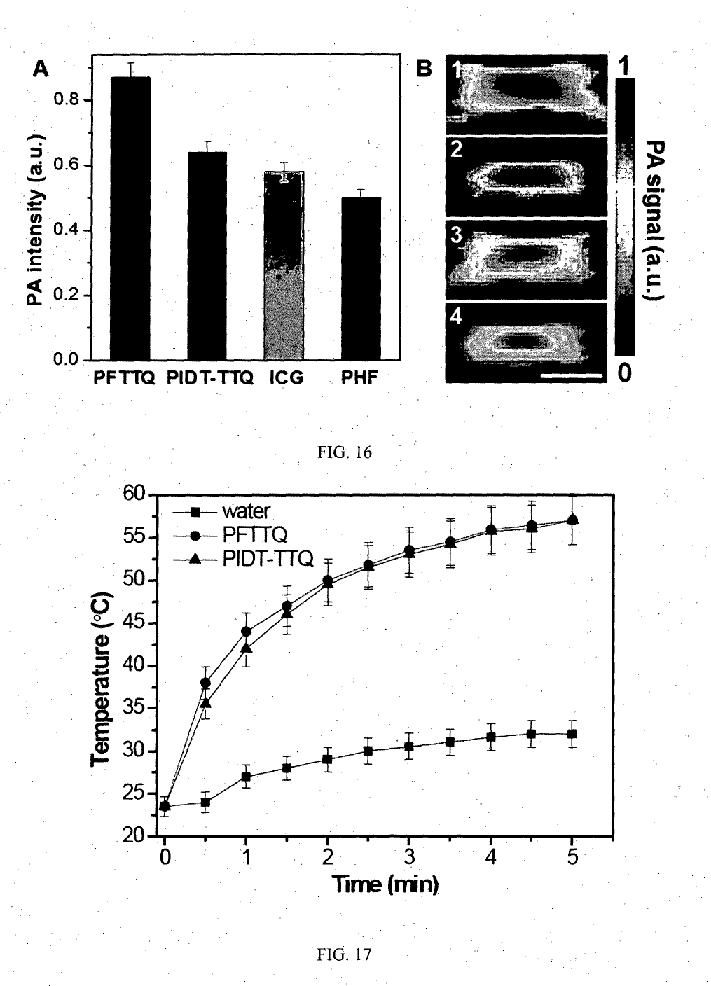

To evaluate the PA signal generation capability of both PFTTQ and PIDT-TTQ NPs, their PA intensities were investigated on a nonabsorbing polyethylene tube with a 50-MHz dark field confocal PAM system, and compared with the PA intensities of ICG and C60, two typical PA contrast agents. The schematic illustration of this setup is shown in FIG. 15. The pulled tubing was filled with the venous samples in the focusing depth. The laser was pulsed with a pulse repetition rate of 10 Hz and coupled by a lens to an optical fiber to illuminate samples. PA waves were detected with a 50-MHz transducer and then through the A/D card to the PC for further data analysis. An optical parametric oscillator pumped by a frequency-tripled Nd:YAG Q-switched laser were employed to provide ˜4 ns laser pulses at a pulse repetition rate of 10 Hz.[35] To make the comparison more reasonable, their concentrations were adjusted to be 1 mg/mL in water. The laser wavelength used for all samples was 800 nm. The PA intensity of PFTTQ NPs is about 1.5-fold higher than that of ICG and 1.74-fold higher than that of PHF. Additionally, the PA intensity of PIDT-TTQ NPs is also better than that of both ICG and PHF. The large light absorption ability of both PFTTQ NPs and PIDT-TTQ NPs as compared to that of ICG and PHF should contribute to the enhancement of PA intensities of CP NPs.

### Example 13

### Photothermal Therapy Investigation

To evaluate the potential of both PFTTQ NPs and PIDT-TTQ NPs as photothermal reagents, a suspension of NPs at 0.5 mg/mL was exposed to 800 nm NIR laser at a power density of 1.5 W/cm² for 5 min. The temperature evolution of pure water under the same condition was also investigated as the control. An obvious temperature increase from 25° C. to 57° C. was observed for both PFTTQ and PIDT-TTQ NP suspensions under laser irradiation as shown in FIG. 17. On the other hand, the temperature of pure water is only slightly increased from 25° C. to 30° C. upon laser irradiation within the same time. The heat generation capacity of CP NPs results from their large absorption coefficients.

### Example 14

### In Vitro Photothermal Therapy

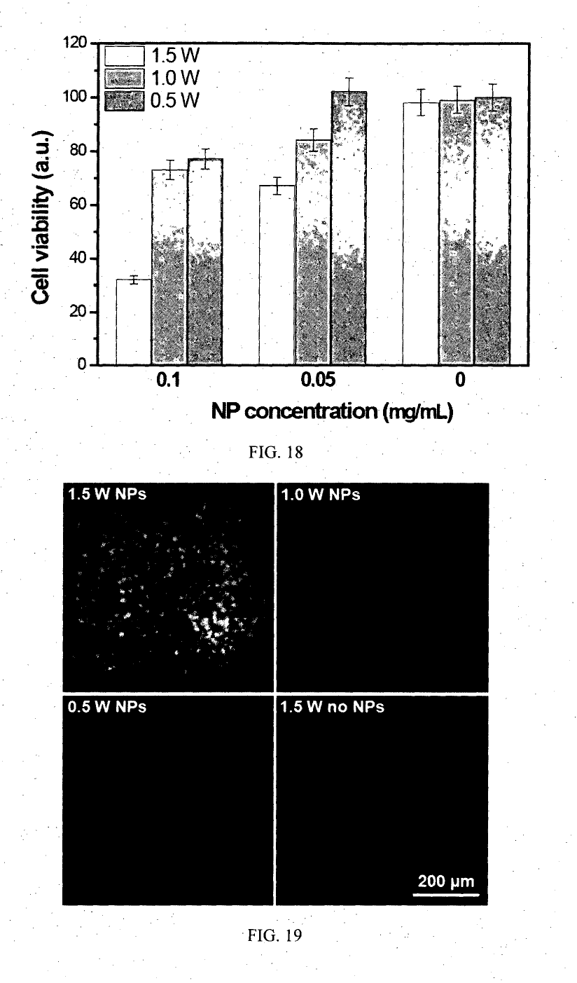

After verifying the photothermal effect of CP NPs, PFTTQ NP was chosen as a typical example to investigate their performance in in vitro environment. MCF-7 breast cancer cells were incubated with PFTTQ NPs for 4 h and then washed with 1× phosphate buffer solution (PBS) twice. Then the washed cells were irradiated with the 808 laser at different laser power densities. After laser irradiation, the cell viabilities were evaluated by the standard methyl thiazolyl tetrazolium (MTT) assay. As shown in FIG. 18, the viabilities of MCF-7 cancer cells without NP incubation do not show obvious decrease upon laser irradiation. However, 26% and 70% of MCF-7 cancer cells were killed upon incubated with 0.05 mg/mL and 0.1 mg/mL PFTTQ NPs, respectively, under 1.5 W/cm² laser irradiation. These results demonstrate that PFTTQ NPs can generate heat efficiently upon NIR laser irradiation, which is ideal for photothermal therapy application.

In addition to the investigation of photothermal effect via MTT assay, the MCF cancer cells were also stained with propidium iodide (PI) after laser irradiation to identify the dead cells. After staining with PI for 30 min, the irradiated cells were imaged with a fluorescence microscopy. Compared to cells that were not incubated with PFTTQ NPs, more cells that had been incubated with PFTTQ NPs were killed with increasing laser power density from 0.5 W/cm² to 1.5 W/cm² as shown in FIG. 19. The cells with PFTTQ NP incubation are mostly destroyed after laser exposure at a power density of 1.5 W/cm². In contrast, MCF-7 cancer cells without NP incubation were not affected after laser irradiation at the power density of 1.5 W/cm². This comparison further illustrates the efficient thermal generation of PFTTQ NPs.

### REFERENCES

1. Frangioni, J. V. Curr. Opin. Biol. 2003, 7, 626.
2. Li, K. et al. Adv. Funct. Mater. 2009, 19, 3535.
3. Kim, C. et al. Chemical Reviews 2010, 110, 2756.
4. Ntziachristos, V. et al. Chemical Reviews 2010, 110, 2783.
5. Wang, L. V. Nature Photonics 2009, 3, 503.
6. Wang, L. H. V. et al. Science 2012, 335, 1458.
7. Xu, M. H. et al. Review of Scientific Instruments 2006, 77.
8. Ntziachristos, V. Nature Methods 2010, 7, 603.
9. Wang, X. D. et al. Optics Letters 2004, 29, 730.
10. Liao, L. D et al. Neuroimage 2010, 52, 562.
11. Liao, L. D.; et al. J. Cerebral Blood Flow and Metabolism 2012, 32, 938.
12. Wang, X. D. et al. Nature Biotechnology 2003, 21, 803.
13. Zhang, H. F. et al Nature Biotechnology 2006, 24, 848.
14. Li, M. L. et al. Proceedings of the Ieee 2008, 96, 481.
15. Kim, C.; et al. Acs Nano 2010, 4, 4559.
16. Kim, G. et al; Journal of Biomedical Optics 2007, 12.
17. Liang, F. et al. Current Medicinal Chemistry 2010, 17, 10.
18. Moon, G. D. et al. J. Am. Chem. Soc. 2011, 133, 4762.
19. Cote, L. J. et al. Am. Chem. Soc. 2009, 131, 11027.
20. Huynh, E. et al. J. Am. Chem. Soc. 2012, 134, 16464.
21. Filonov, G. S. et al.; Angew. Chem. Int. Ed. 2012, 51, 1448.
22. de la Zerda, A.; et al. Nano Letters 2010, 10, 2168.
23. Ku, G.; et al. Optics Letters 2005, 30, 507.
24. Thomas, S. W. et al. Chem. Rev. 2007, 107, 1339.
25. McQuade, D. T. et al. Chem. Rev. 2000, 100, 2537.
26. Liu, B.; et al. Chem. Mater. 2004, 16, 4467.
27. Duarte, A.; et al. Chem. Mater. 2011, 23, 501.
28. Wu, et al. Angew. Chem. Int. Ed. 2011, 50, 1.
29. Lee, S. H., et al., Macromolecules 2011, 44, 4742
30. Drake, J. M., et al., Chem. Phys. Lett., 113, (1985), 530-534.
31. Yang, R. Q.; et al., J. Am. Chem. Soc. 2006, 128, 16532.
32. Prashant, C., et al, Biomaterials, 31, (2010), 5588-5597.
33. van Vlerken, L. E., et al., Mol. Pharmaceuticals, 5, (2008), 516-526.
34. Berlier, J. E., et al., P. J. Histochem. Cytochem. 2003, 51, 1699.
35. Liao, L. D. et al. Journal of Biomedical Optics 2012, 17.

The teachings of all patents, published applications and references cited herein are incorporated by reference in their entirety.

While this invention has been particularly shown and described with references to example embodiments thereof, it will be understood by those skilled in the art that various changes inform and details may be made therein without departing from the scope of the invention encompassed by the appended claims.

1. A conjugated polymer of formula (I): 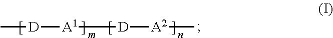
wherein:
D is a conjugated system of one or more optionally substituted aromatic or heteroaromatic groups comprising: 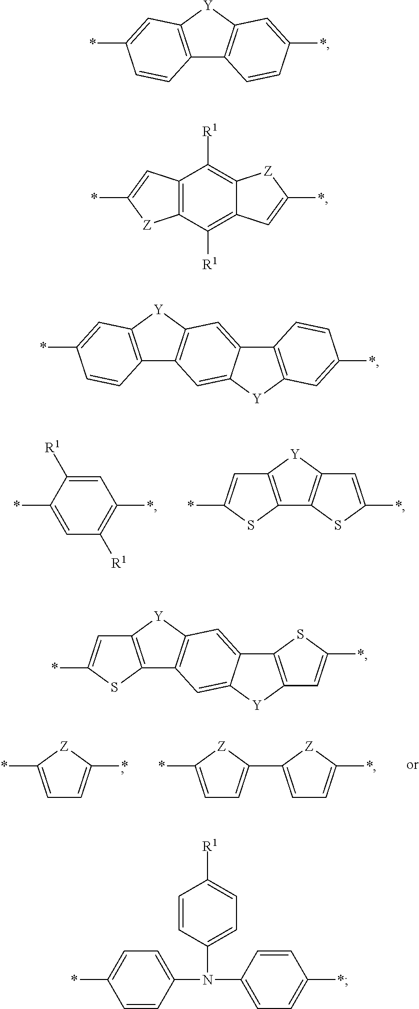
A¹ is a conjugated system of one or more optionally substituted aromatic or heteroaromatic groups;
A² is a conjugated system of one or more optionally substituted aromatic or heteroaromatic groups;
m is an integer from 1 to 100;
n is an integer from 0 to 100;
Y is C(R¹)₂, Si(R¹)₂, or NR¹;
Z is O, S or Se;
each R¹ is independently H, (CH₂)ₚQ, (OCH₂CH₂)ₚQ 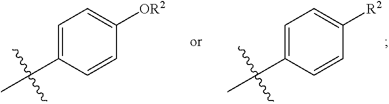
p is an integer ranging from 1 to 24;
R² is (C₁-C₁₂)alkyl, (C₆-C₁₄)aryl, or (C₁-C₁₀)alkoxy(C₁-C₁₂)alkyl;
Q is independently CH₃, H, COOH, NH₂, NH₃⁺, N₃, SH, SO₃Na, PO₃Na, or 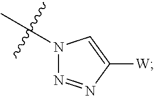
W is independently a fluorophore, a bioconjugate, a (C₁-C₁₀₀)alkyl group bound to a fluorophore or a bioconjugate, or a polyethyleneoxide group bound to a fluorophore or a bioconjugate;
* is the point of attachment to A¹, A², or a polymeric unit;
for n greater than or equal to 1, the energy band gap of A¹ is larger than the energy band gap of A²; and
further wherein the energy band gap of D is larger than the energy band gap of A¹ and the energy band gap of A².

2. The conjugated polymer of claim 1, wherein the conjugated polymer is assembled into a conjugated polymer-based nanoparticle.

3. The conjugated polymer of claim 1, wherein the conjugated polymer is encapsulated into a biocompatible matrix comprising polyethylene glycol, polyethylene glycol conjugated to 1,2-distearoyl-sn-glycero-3-phosphoethanolamine, bovine serum albumin (BSA) protein, poly(lactic-co-glycolic acid) (PLGA) block copolymers, collagens or lipids.

4. (canceled)

5. The conjugated polymer of claim 1, wherein A¹ and A² each optionally and independently comprise: 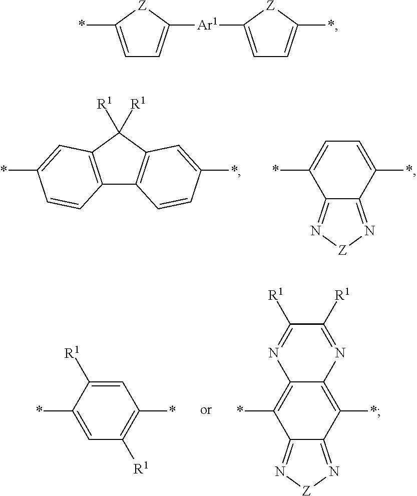
wherein:
Ar¹ is 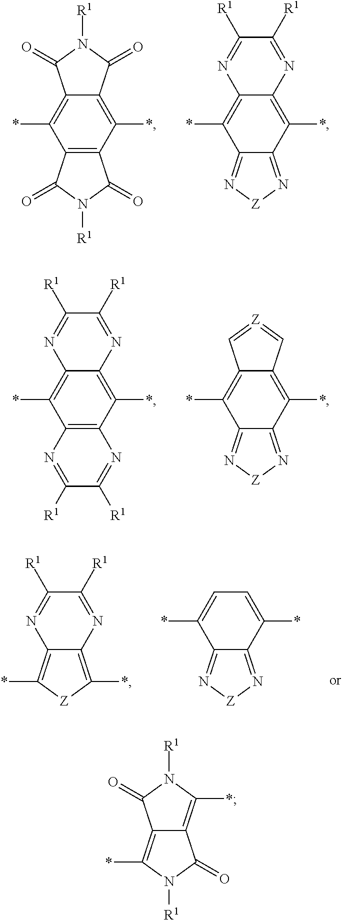
and
R¹ and Z are as defined in claim 1.

6. The conjugated polymer of claim 1, wherein W comprises: 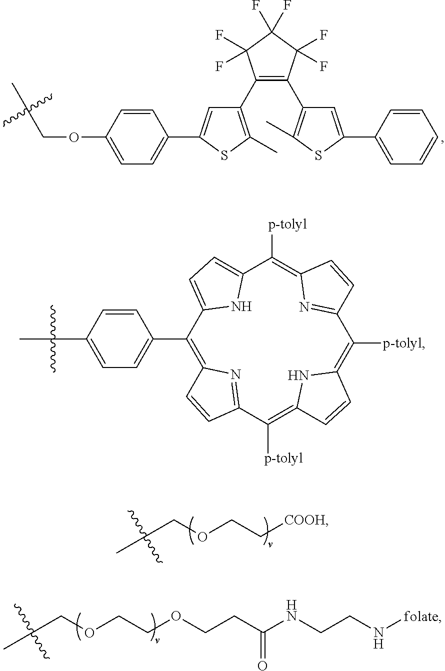
an amine-labeled cyclic peptide, an oligonucleotide, an acyclic peptide, or a protein;
wherein v is an integer ranging from 0 to 45.

7. The conjugated polymer of claim 1, wherein D is

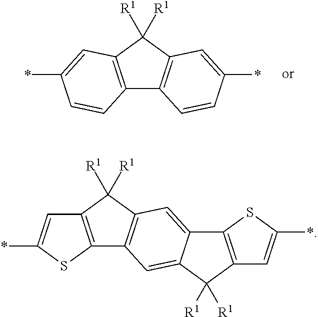

8. The conjugated polymer of claim 1, having the structure: 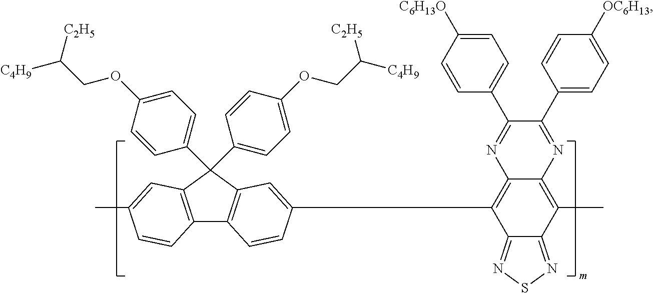
wherein
m is an integer from 3 to 100.

9. The conjugated polymer of claim 1, having the structure: 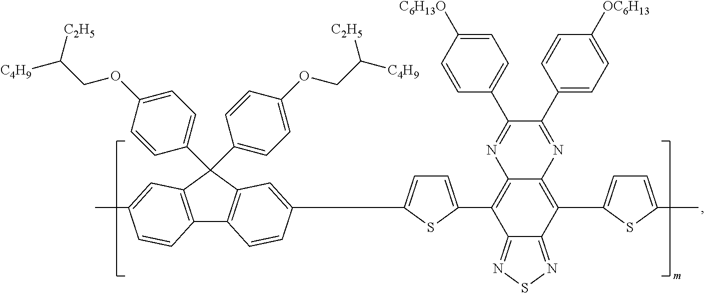
wherein
m is an integer from 4 to 100.

10. The conjugated polymer of claim 1, having the structure: 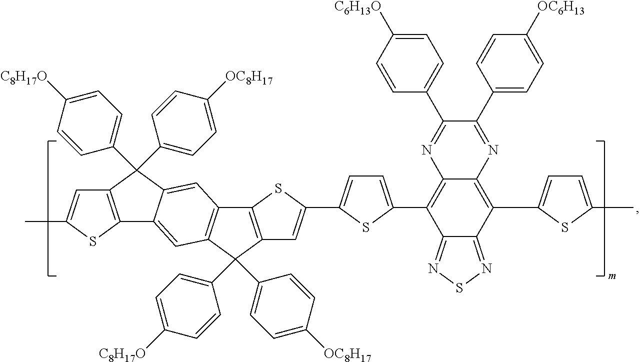
wherein
m is an integer from 4 to 100.

11. The conjugated polymer of claim 1, wherein:
D is; 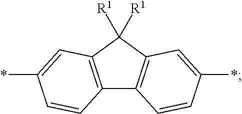
R¹ is H, (CH₂)ₚQ, (OCH₂CH₂)ₚQ, 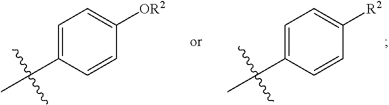
p is an integer ranging from 1 to 24;
R² is (C₁-C₁₂)alkyl, (C₆-C₁₄)aryl, or (C₁-C₁₀)alkoxy(C₁-C₁₂)alkyl;
Q is CH₃, H, COOH, NH₂, NH₃⁺, N₃, SH, SO₃Na, PO₃Na, 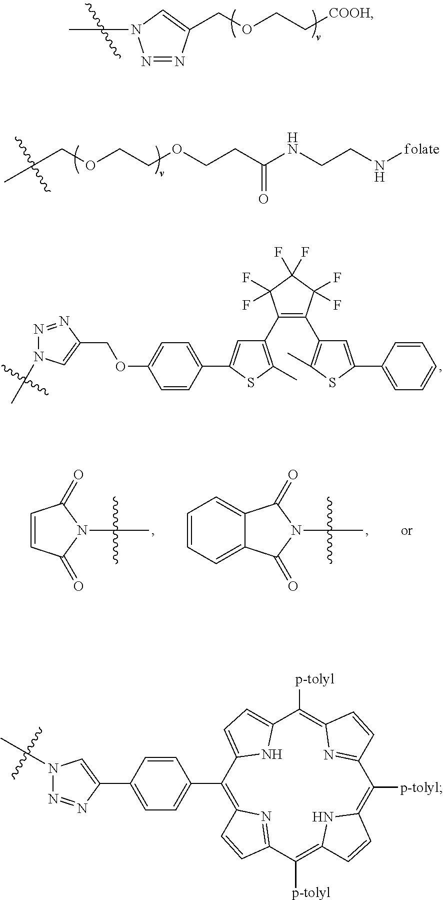
and
v is an integer ranging from 0 to 45.

12. A conjugated molecule of formula (II): 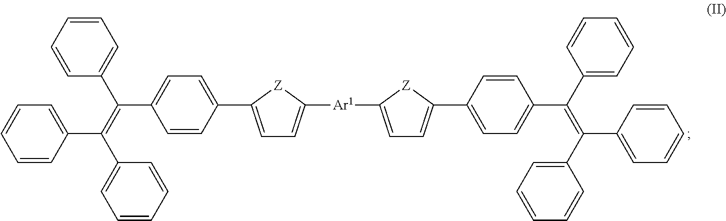
wherein:
Ar¹ is a polycyclic carbocyclic or heterocyclic aromatic group;
Z is independently O, Se or S.

13. The conjugated molecule of claim 12, wherein:
Ar¹ is 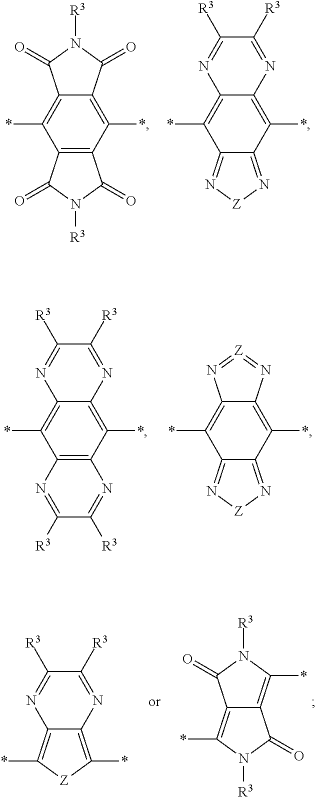
R³ is independently H, (CH₂)ₚCH₃, (OCH₂CH₂)ₚCH₃, 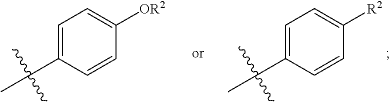
p is an integer ranging from 1 to 24;
R² is (C₁-C₁₂)alkyl, (C₆-C₁₄)aryl, or (C₁-C₁₀)alkoxy(C₁-C₁₂)alkyl; and
* is the point of attachment to A¹, A², or a polymeric unit.

14. The conjugated molecule of claim 13, wherein Ar¹ is

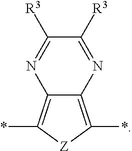

15. A method for making conjugated polymer-based nanoparticles, comprising:
a) reacting an cross coupling partner of formula J-D-J;
wherein D is 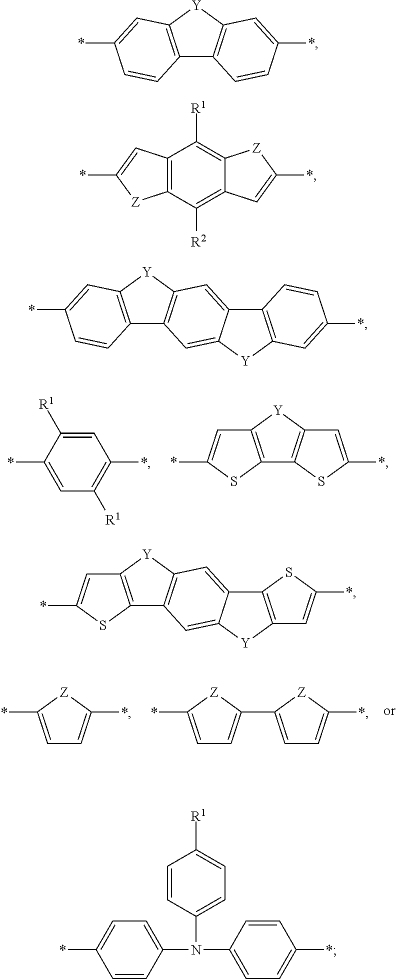
J is independently B(OH)₂, B(pin), B(cat), BF₃K, B(O(CH₂)₃O) or Sn(C₁-C₄ (alkyl))₃;
Y is C(R¹)₂, Si(R¹)₂, or NR¹;
Z is O, S or Se;
each R¹ is independently H, (CH₂)ₚQ, (OCH₂CH₂)ₚQ, 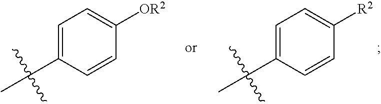
p is an integer ranging from 1 to 24;
R² is (C¹-C₁₂)alkyl, (C₆-C₁₄)aryl, or (C¹-C₁₀)alkoxy(C₁-C₁₂)alkyl;
Q is independently CH₃, H, COOH, NH₂, NH₃⁺, N₃, SH, SO₃Na, or PO₃Na;
with a dihalide containing A¹, and, for conjugated polymers of formula (I) for which n is not equal to zero, a second dihalide containing A²;
A¹ is a conjugated system of one or more optionally substituted aromatic or heteroaromatic groups;
A² is a conjugated system of one or more optionally substituted aromatic or heteroaromatic groups;
in the presence of a palladium (0), palladium (II), nickel (0) or nickel (II) catalyst;
to produce a conjugated polymer of formula (I) 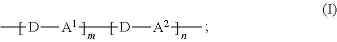
m is an integer from 1 to 100;
n is an integer from 0 to 100;
b) adding the conjugated polymer of formula (I) from step a) that is solubilized or suspended in at least one organic solvent into aqueous solution, enabling the self-assembly of the conjugated polymers into nanoparticles.

16. (canceled)

17. A conjugated polymer of claim 1, wherein the conjugated polymer is loaded onto a 1,2-distearoyl-sn-glycero-3-phosphoethanolamine-N-[methoxy(polyethylene glycol)] (DSPE-PEG) lipid.

18. (canceled)

19. The method of claim 15, wherein for compounds in which Q is N₃, the method further comprises an additional step, the step being performed after step a) and before step b), the step comprising:
Reacting a conjugate polymer of formula (III) produced in step a) 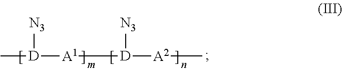
with an alkyne of formula (IV) 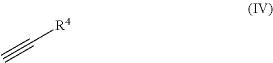
wherein R⁴ is selected from 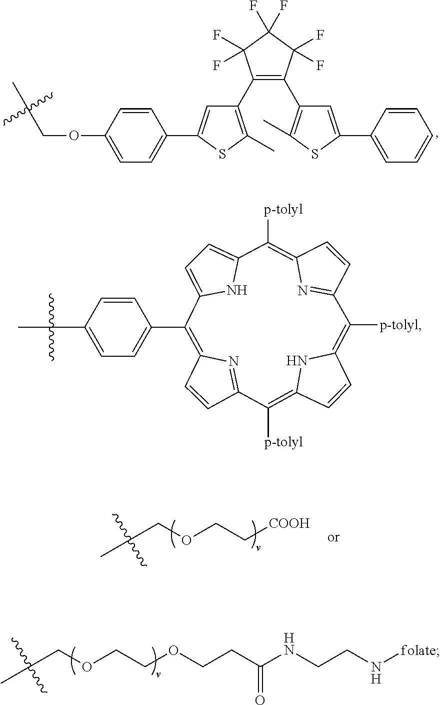
v is an integer ranging from 0 to 45;
via click chemistry to produce a compound of the formula (V) 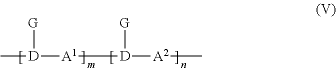
wherein each G is independently selected from N₃ or

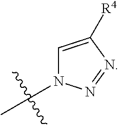

20. A method of using a conjugated polymer nanoparticle of claim 2 in any one of the following applications:
as a fluorescent probe for a bioimaging and biosensing application;
in cancer cell staining;
photothermal ablation of a cell;
photothermal ablation of a cancer cell;
molecular or nanoscale probe for detecting biological species in vivo;
as a photosensitizer or therapeutic agent in photodynamic therapy; or
a photoacoustic imaging contrast agent.

21. (canceled)

22. A conjugated polymer nanoparticle of claim 2, wherein R¹ is (CH₂)ₚQ and Q is selected from N₃ or 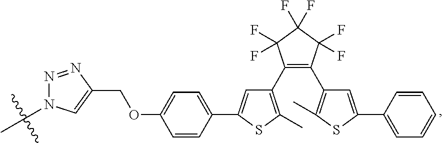 [SMILES: *n1cc(COc2ccc(-c3cc(C4=C(c5cc(-c6ccccc6)sc5C)C(F)(F)C(F)(F)C4(F)F)c(C)s3)cc2)nn1]
wherein at least one Q is

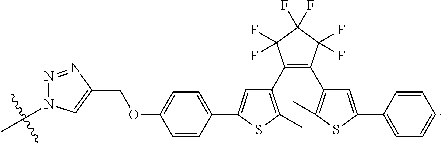 [SMILES: *n1cc(COc2ccc(-c3cc(C4=C(c5cc(-c6ccccc6)sc5C)C(F)(F)C(F)(F)C4(F)F)c(C)s3)cc2)nn1]

23. (canceled)

24. A conjugated polymer nanoparticle of claim 2, wherein R¹ is (CH₂)ₚQ or (OCH₂CH₂)ₚQ and Q is selected from N₃ 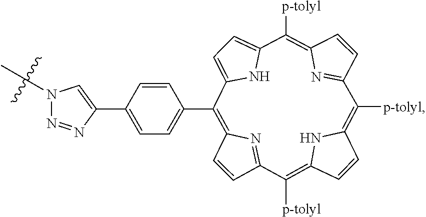
wherein at least one Q is

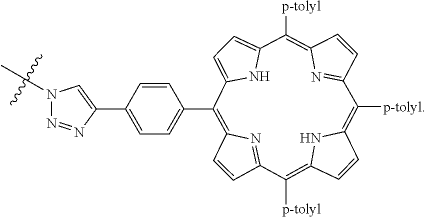

25-28. (canceled)

29. A method for photoacoustic imaging of a target, comprising:
a) incubating a target with a polymer of Formula (I) according to claim 1 to form an incubated mixture;
b) irradiating the mixture of step a) with a pulsed laser, wherein the pulsed laser optically excites the polymer, to generate thermally-induced acoustic waves, wherein the acoustic waves result from energy emission from the excited polymer;
c) detecting the thermally-induced acoustic waves of step b) with ultrasound; and
d) translating the acoustic waves detected by ultrasound into an image of the target.

30-32. (canceled)
